SEQUENCE LISTING (13 sequences)

SEQ ID NO: 1 - protein, 856 aa, synthetic construct
  REGION 1..856: note = Synthetic Polypeptide
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 2 - protein, 849 aa, synthetic construct
  REGION 1..849: note = Synthetic Polypeptide
MKVMGTKKNYQHLWRWGIMLLGMLMMSSAAEQLWVTVYYGVPVWREANTTLFCASDAKAYDTEVHNVWATHACVPTDPNPQEVVMGNVTEDFNMWKNNMVEQMHEDIISLWDQSLKPCVKLTPLCVTLHCTNVTISSTNGSTANVTMREGMKNCSFNTTTVIRDKIQKEYALFYKLDIVPIEGKNTNTSYRLINCNTSVITQACPKVSFEPIPIHYCAPAGFAILKCNNKTFNGKGPCRNVSTVQCTHGIKPVVSTQLLLNGSLAEEDIIIRSENFTNNGKNIIVQLKEPVKINCTRPGNNTRRSINIGPGRAFYATGAIIGDIRKAHCNISTEQWNNTLTQIVDKLREQFGNKTIIFNQSSGGDPEVVMHTFNCGGEFFYCNSTQLFNSTWFNNGTSTWNSTADNITLPCRIKQVINMWQEVGKAMYAPPIRGQIDCSSNITGLILTRDGGSNSSQNETFRPGGGNMKDNWRSELYKYKVVKIEPLGIAPTRAKRRVVQREKRAVTLGAVFLGFLGAAGSTMGAASLTLTVQARLLLSGIVQQQSNLLRAIEAQQHMLQLTVWGIKQLQARVLAIERYLKDQQLLGIWGCSGKLICTTTVPWNTSWSNKSYDYIWNNMTWMQWEREIDNYTGFIYTLIEESQNQQEKNELELLELDKWASLWNWFNITNWLWYIKLFIMIIGGLVGLRTVCAVLSIVNRVRQGYSPLSFQTRLPNPRGPGRPEETEGEGGERDRDRSARLVNGFLAIIWDDLRSLCLFSYHRLRDLLLIVARVVEILGRRGWEILKYWWNLLKYWSQELKNSAVSLLNVTAIAVAEGTDRVIEIVQRAVRAILHIPTRIRQGFERALL

SEQ ID NO: 3 - protein, 858 aa, synthetic construct
  REGION 1..858: note = Synthetic Polypeptide
MRVRGIPRNWPQWWIWGILGFWMIIICRVVGNLDLWVTVYYGVPVWKEAKTTLFCASDAKAYDKEVHNVWATHACVPTDPNPQEIVLENVTENFNMWKNDMVDQMHEDIISLWDQSLKPCVKLTPLCVTLNCKNVNISANANATATLNSSMNGEIKNCSFNTTTELRDKKQKVYALFYKPDVVPLNGGEHNETGEYILINCNSSTITQACPKVSFDPIPIHYCAPAGYAILKCNNKTFNGTGPCNNVSTVQCTHGIKPVVSTQLLLNGSLAEEEIIVRSENLTNNIKTIIVHLNKSVEIKCTRPNNNTRKSVRIGPGQTFYATGEIIGDIREAHCNISRETWNSTLIQVKEKLREHYNKTIKFEPSSGGDLEVTTHSFNCRGEFFYCDTTKLFNETKLFNESEYVDNKTIILPCRIKQIINMWQEVGRAMYAPPIEGNITCKSNITGLLLTWDGGENSTEGVFRPGGGNMKDNWRSELYKYKVVEIKPLGVAPTKSKRKVVGREKRAVGLGAVLLGFLGAAGSTMGAASITLTVQARQLLSGIVQQQSNLLRAIEAQQHLLQLTVWGIKQLQTRVLAIERYLKDQQLLGLWGCSGKLICATAVPWNSSWSNKSLGDIWDNMTWMQWDREISNYTNTIFRLLEDSQNQQEKNEKDLLALDSWKNLWNWFDITNWLWYIKIFIMIVGGLIGLRIIFGVLAIVKRVRQGYSPLSFQTLIPNPRGPDRLGRIEEEGGEQDKDRSIRLVSGFLALAWDDLRSLCLFSYHQLRDFILTAARAAELLGRSSLRGLQRGWEVLKYLGNLVQYWGLELKRSAINLFDTIAIAVAEGTDRIIEVIQRICRAIRYIPTRIRQGFEAALL

SEQ ID NO: 4 - protein, 860 aa, synthetic construct
  REGION 1..860: note = Synthetic Polypeptide
MRVMGIQRNCQHLFRWGTMILGMIIICSAAENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNNNTRKSIRIGPGQAFYATGDIIGDIRQAHCTVSKATWNETLGKVVKQLRKHFGNNTIIRFANSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRAKRRVVGREKRAVGIGAVFLGFLGAAGSTMGAASMTLTVQARNLLSGIVQQQSNLLRAIEAQQHLLKLTVWGIKQLQARVLAVERYLRDQQLLGIWGCSGKLICTTNVPWNSSWSNRNLSEIWDNMTWLQWDKEISNYTQIIYGLLEESQNQQEKNEQDLLALDKWASLWNWFDISNWLWYIKIFIMIVGGLIGLRIVFAVLSVIHRVRQGYSPLSFQTHTPNPRGLDRPERIEEEDGEQDRGRSTRLVSGFLALAWDDLRSLCLFCYHRLRDFILIAARIVELLGHSSLKGLRLGWEGLKYLWNLLAYWGRELKISAINLFDTIAIAVAEWTDRVIEIGQRLCRAFLHIPRRIRQGLERALL

SEQ ID NO: 5 - protein, 500 aa, synthetic construct
  REGION 1..500: note = Synthetic Polypeptide
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 6 - DNA, 4307 bp, synthetic construct
  misc_feature 1..4307: note = Synthetic Polynucleotide
  source 1..4307: mol_type = other DNA
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 7 - protein, 99 aa, synthetic construct
  REGION 1..99: note = Synthetic Polypeptide
PQITLWKRPLVTIKIGGQLKEALLDTGADDTVLEEMNLPGRWKPKMIGGIGGFIKVRQYDQILIEICGHKAIGTVLVGPTPVNIIGRNLLTQIGCTLNF

SEQ ID NO: 8 - protein, 794 aa, synthetic construct
  REGION 1..794: note = Synthetic Polypeptide
MELRPWLLWVVAATGTLVLLAADAQGQKVFTNTWAVRIPGGPAVANSVARKHGFLNLGQIFGDYYHFWHRGVTKRSLSPHRPRHSRLQREPQVQWLEQQVAKRRTKRDVYQEPTDPKFPQQWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMVALAQNWTTVAPQRKCIIDILTEPKDIGKRLEVRKTVTACLGEPNHITRLEHAQARLTLSYNRRGDLAIHLVSPMGTRSTLLAARPHDYSADGFNDWAFMTTHSWDEDPSGEWVLEIENTSEANNYGTLTKFTLVLYGTAPEGLPVPPESSGCKTLTSSQACVVCEEGFSLHQKSCVQHCPPGFAPQVLDTHYSTENDVETIRASVCAPCHASCATCQGPALTDCLSCPSHASLDPVEQTCSRQSQSSRESPPQQQPPRLPPEVEAGQRLRAGLLPSHLPEVVAGLSCAFIVLVFVTVFLVLQLRSGFSFRGVKVYTMDRGLISYKGLPPEAWQEECPSDSEEDEGRGERTAFIKDQSAL

SEQ ID NO: 9 - protein, 668 aa, synthetic construct
  REGION 1..668: note = Synthetic Polypeptide
MDAMKRGLCCVLLLCGAVFVSPSQEIHARFRRGARAENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLICNTSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNNNTRKSIRIGPGQAFYATGDIIGDIRQAHCNVSKATWNETLGKVVKQLRKHFGNNTIIRFANSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 10 - RNA, 47 nt, synthetic construct
  misc_feature 1..47: note = Synthetic Polynucleotide
  source 1..47: mol_type = other RNA
gggaaataagagagaaaagaagagtaagaagaaatataagagccacc

SEQ ID NO: 11 - RNA, 57 nt, synthetic construct
  misc_feature 1..57: note = Synthetic Polynucleotide
  source 1..57: mol_type = other RNA
gggaaataagagagaaaagaagagtaagaagaaatataagaccccggcgccgccacc

SEQ ID NO: 12 - RNA, 119 nt, synthetic construct
  misc_feature 1..119: note = Synthetic Polynucleotide
  source 1..119: mol_type = other RNA
tgataataggctggagcctcggtggccatgcttcttgccccttgggcctccccccagcccctcctccccttcctgcacccgtacccccgtggtctttgaataaagtctgagtgggcggc

SEQ ID NO: 13 - RNA, 119 nt, synthetic construct
  misc_feature 1..119: note = Synthetic Polynucleotide
  source 1..119: mol_type = other RNA
tgataataggctggagcctcggtggcctagcttcttgccccttgggcctccccccagcccctcctccccttcctgcacccgtacccccgtggtctttgaataaagtctgagtgggcggc

(19) United States
(12) Patent Application Publication
(10) Pub. No.: US 2025/0073327 A1
(43) Pub. Date: Mar. 6, 2025

(54) HIV RNA VACCINES

(71) Applicants: ModernaTX, Inc., Cambridge, MA (US); The United States of America, as represented by the Department of Health and Human Services, Rockville, MD (US)

(72) Inventors: Paolo Lusso, Rockville, MD (US); Peng Zhang, Rockville, MD (US); Elisabeth Narayanan, Cambridge, MA (US); Sayda Mahgoub Elbashir, Cambridge, MA (US)

(73) Assignees: ModernaTX, Inc., Cambridge, MA (US); The United States of America, as represented by the Department of Health and Human Services, Rockville, MD (US)

(21) Appl. No.: 18/771,978

(22) Filed: Jul. 12, 2024

Related U.S. Application Data
(62) Division of application No. 17/439,198, filed on Sep. 14, 2021, now Pat. No. 12,070,495.
(60) Provisional application No. 62/819,394, filed on Mar. 15, 2019.

(51) Int. Cl. A61K 39/21; A61K 39/00; A61K 39/39

(52) U.S. Cl. CPC A61K 39/21; A61K 39/39; A61K 2039/53; A61K 2039/55516; A61K 2039/6018

(57) ABSTRACT

Provided herein are methods and compositions for inducing in a subject a broad neutralizing antibody response to human immunodeficiency virus (HIV) infection.

### RELATED APPLICATION

This application is a national stage filing under 35 U.S.C. § 371 of International Application No. PCT/US2020/022710, filed Mar. 13, 2020, which claims the benefit under 35 U.S.C. § 119(e) of U.S. Provisional Application No. 62/819,394, filed Mar. 15, 2019, each of which is incorporated by reference herein in its entirety.

### BACKGROUND

Human Immunodeficiency Virus (HIV) is a lentivirus that primarily infects hosts and cells through communication of bodily fluids or through pregnancy. HIV infects cells essential to the immune system, such as CD4+ T cells, macrophages, and dendritic cells, ultimately causing cell death. When the rate and magnitude of cell death causes essential cell levels to fall below critical levels, it become increasingly more difficult for the host to mount an effective immune response, leading to acquired immunodeficiency syndrome (AIDS). Without treatment, the average survival time after infection with HIV is estimated to be 9 to 11 years.

### SUMMARY

Provided herein are mRNA-based immunogenic compositions (e.g., vaccines) and therapies that elicit neutralizing antibodies against a broad range of human immunodeficiency virus (HIV) (e.g., HIV-1) strains circulating worldwide. Use of mRNA as a vehicle for eliciting broadly neutralizing antibodies against multiple strains of HIV has the advantage of persistent in vivo expression of endogenous proteins (e.g., identical to those found in nature). Further, the co-formulation used herein, with mRNA encoding HIV Env and mRNA encoding lentivirus Gag, enables in vivo production of extracellular virus-like particles (VLPs), mimicking natural infection. Further still, the use in some embodiments of mRNA encoding full-length or partially truncated membrane-bound HIV Env gp160 or gp150 provides a bona fide “native” confirmation of expressed Env, unlike truncated soluble Env SOSIP trimers. Other embodiments provided herein provide mRNA-based immunogenic compositions that further include mRNA encoding the viral protease that is used for Gag processing, and/or mRNA encoding furin, which is used for Env processing. The intensive immunization protocols provided herein mimic continuous, high-level antigenic load in HIV-infected patients who develop broad neutralizing antibodies against HIV, and the multiple-strain heterologous boost dosing focuses the antibody response on “common” broad neutralizing antibody epitopes to the exclusion of strain-specific epitopes. In some embodiments, complete filling of Env glycan holes provides the additional advantage of an exclusion of dominant antibody responses against vaccine-irrelevant epitopes.

Induction of neutralizing antibodies with a broad spectrum of action against heterologous tier-2 isolates is a key requirement for a bonafide protective HIV-1 vaccine. Yet, none of the vaccine strategies hitherto devised has achieved this objective. In the studies presented herein, an intensive HIV-1 envelope (Env)-based immunization scheme was tested in rhesus macaques. Four groups of animals were immunized with coformulated HIV-1 Env (WITO gp150, Clade B) and SIV Gag (from SIVmac239) in order to promote the in vivo formation and release of virus-like particles. Two groups of animals received wild-type Env and two received interdomain-stabilized Env bearing the amino acid 113-432 disulfide bridge (Zhang et al., Cell Host & Microbe 2018; 23: 832); two groups received mRNA only, while two received mRNA followed by protein boost with the autologous SOSIP trimer. All groups were subsequently boosted with mRNA expressing Env from two heterologous HIV-1 isolates (BG505, Clade A, and DU422, Clade C): again, two groups received autologous protein boosts. High titers of neutralizing antibodies against the autologous Env (WITO.27) or against tier-1a viruses were readily induced after the second autologous immunization, becoming increasingly more persistent after each booster injection. Following the third heterologous boost, low titers of neutralizing antibodies against heterologous tier-2 viruses of different Clades were detected, including JR-FL and 12 Envs of different Clades derived from the reference small global panel. Further boosting with either mRNA or protein increased both the magnitude and durability of cross-Clade tier-2 heterologous NAb titers. Live virus challenges have been performed using repeated low-dose mucosal inoculation of a heterologous tier-2 SHIV (AD8) resulting in complete protection or delayed infection. These results provide evidence for the elicitation of cross-Clade tier-2 heterologous broadly neutralizing antibodies by mRNA immunization in a preclinical vaccine model.

In some aspects, the present disclosure provides methods of inducing in a human subject an immune response to HIV (e.g., HIV-1), the methods comprising (a) during a first period of time, administering to a subject an initial dose and multiple autologous boost doses of an HIV mRNA vaccine comprising mRNA encoding an HIV envelope (Env) protein and a mRNA encoding a lentivirus group-specific antigen (Gag) protein formulated in a lipid nanoparticle, (b) during a second period of time, administering to the subject multiple heterologous boost doses of an HIV mRNA vaccine comprising mRNA encoding an HIV Env protein and a mRNA encoding a lentivirus Gag protein formulated in a lipid nanoparticle, and (c) producing in the subject a broad and potent neutralizing antibody response against multiple strains of HIV.

In other aspects, the methods comprise, in addition to mRNA encoding Env and Gag, mRNA encoding one or both of two enzymes that are used for the full proteolytic processing of Gag and Env, respectively, and thereby promote a more efficient production and release of properly formed virus-like particles. The two enzymes are: i) the viral protease of HIV-1 or SIV, for Gag processing, and furin, a host enzyme for Env processing.

In some embodiments, the broad and potent neutralizing antibody response comprises the production of neutralizing antibodies that bind to shared epitopes on Env proteins from the multiple strains of HIV, including multiple tier-2 strains from different Clades. In some embodiments, the broadly neutralizing antibody response has an ID50 titer of greater than 20 or greater than 50.

In some embodiments, the first period of time is 1-30 weeks following administration of the initial dose of the HIV mRNA vaccine. In some embodiments, the second period of time is 30-60 weeks following administration of the initial dose of the HIV mRNA vaccine.

In some embodiments, the time between any two doses of the HIV mRNA vaccine of (a) and/or (b) is at least 1 week. In some embodiments, the time between any two doses of the HIV mRNA vaccine of (a) and/or (b) is at least 4 weeks. In some embodiments, the time between any two doses of the HIV mRNA vaccine of (a) and/or the HIV mRNA vaccine of (b) is 4-10 weeks.

In some embodiments, the ratio of mRNA encoding an HIV Env protein to the mRNA encoding a lentivirus Gag protein in the HIV mRNA vaccine of (a) and/or (b) is 1:1 to 10:1 (e.g., 1:1, 2:1, 3:1, 4:1, 5:1, 6:1, 7:1, 8:1, 9:1, or 10:1). In some embodiments, the ratio of mRNA encoding an HIV Env protein to the mRNA encoding a lentivirus Gag protein in the HIV mRNA vaccine of (a) and/or (b) is 3:2 to 9:2 (e.g., 3:2, 2:1, 5:2, 3:1, 7:2, 4:1, or 9:2). In some embodiments, the ratio of mRNA encoding an HIV Env protein to the mRNA encoding a lentivirus Gag protein in the HIV mRNA vaccine of (a) and/or (b) is 3:2 or at least 3:2.

In some embodiments, the ratio of mRNA encoding an HIV Env protein to the mRNA encoding furin is 10:1 to 30:1. In some embodiments, the ratio of mRNA encoding an HIV Env protein to the mRNA encoding furin is 10:1, 15:1, 20:1, 25:1, or 30:1. In some embodiments, the ratio of mRNA encoding an HIV Env protein to the mRNA encoding furin is 20:1 or at least 20:1. In some embodiments, the ratio of mRNA encoding a lentivirus Gag protein to the mRNA encoding the viral protease is 30:1 to 50:1. In some embodiments, the ratio of mRNA encoding a lentivirus Gag protein to the mRNA encoding the viral protease is 30:1, 35:1, 40:1, 45:1, or 50:1. In some embodiments, the ratio of mRNA encoding a lentivirus Gag protein to the mRNA encoding the viral protease is 40:1 or at least 40:1.

In some embodiments, an mRNA encoding a lentivirus Gag protein is replaced by an mRNA encoding the full Gag-Pol open reading frame of HIV-1 or SIV, which comprises the viral protease gene that is transcribed after a ribosomal frame-shift, thus providing an alternative to using an mRNA encoding a viral protease. In some embodiments, the Gag-Pol precursor is partially truncated to reduce its length while maintaining the full protease gene.

In some embodiments, the HIV Env protein of the mRNA HIV vaccine of (a) is selected from an HIV Env protein of Group M Clade A-K (e.g., selected from Clade A, Clade AC, Clade AE, Clade AG, Clade B, Clade C, Clade D, and Clade G), wherein the HIV Env protein of the mRNA HIV vaccine of (b) is selected from an HIV Env protein of Group M Clade A-K (e.g., selected from Clade A, Clade AC, Clade AE, Clade AG, Clade B. Clade C, Clade D, and Clade G), and wherein the Clade(s) of the HIV Env protein of (a) is different from the Clade(s) of the HIV Env protein of (b).

In some embodiments, the HIV Env protein of (a) and/or (b) comprises HIV Env SOSIP.664 mutations. In some embodiments, the HIV Env protein of (a) and/or (b) is a membrane-bound HIV Env protein. In some embodiments, the cytosolic portion of the HIV Env protein of (a) and/or (b) is truncated or partially truncated. In some embodiments, the membrane-bound HIV Env protein is gp150, with a truncation of the gp41 cytoplasmic domain at position 745, or full-length gp160. In some embodiments, the HIV Env protein of (a) and/or (b) comprises a sequence of an Env protein of an HIV strain obtained from an infected subject who has broad and potent neutralizing antibodies to HIV Env protein. In some embodiments, the HIV Env protein of (a) and/or (b) comprises a consensus sequence of variants an Env protein of an HIV strain obtained from an infected subject who has broad and potent neutralizing antibodies to HIV Env protein.

In some embodiments, the HIV Env protein of (a) and/or (b) is an uncommon Env capable of directly engaging unmutated common ancestor (UCA) antibodies from the lineages that originated some broad and potent neutralizing antibodies, such as VRC01, CH103, PG9, CHOI, and others. In some embodiments, to facilitate binding to UCA antibodies, the Env protein comprises a mutation selected from 153E, 190G and N276D, relative to strain WITO4160.27 HIV Env protein, or other mutations suitable to remove the glycans at positions 188 and 276, optionally wherein the HIV Env protein of (a) and/or (b) further comprises a disulfide bridge at 113C-432GCG. In some embodiments, the HIV Env protein of (a) and/or (b) further comprises a mutation selected from N460D and N463D relative to strain BG505 HIV Env protein, or other suitable mutations to remove the glycans at positions 460 and 463. In some embodiments, the HIV Env protein of (a) and/or (b) comprises a mutation selected from K295N, D386N, and 375Y, relative to strain DU422.1 HIV Env protein, optionally wherein the HIV Env protein of (a) and/or (b) further comprises a disulfide bridge at 133C-432GCG. In some embodiments, the HIV Env protein of (a) and/or (b) comprises a mutation selected from T322N and S375Y, relative to strain WITO4160.27 HIV Env protein, optionally wherein the HIV Env protein of (a) and/or (b) further comprises a disulfide bridge at 113C-429GCG.

In some embodiments, the HIV Env protein of (a) and/or (b) is a tier-2 Env with all the major glycan holes filled in by insertion of the missing glycans.

In some embodiments, the lentivirus is selected from human immunodeficiency virus (HIV), simian immunodeficiency virus (SIV), and murine leukemia virus (muLV).

In some embodiments, the HIV mRNA vaccine further comprises a Vesicular stomatitis virus (VSV) or VSV-AG core protein, or the lentivirus-derived Gag is replaced by a VSV-AG or VSV core protein. Viral proteins include proteins that are capable of self-assembling into the VLP (Freed, E. O., J. Virol., 76, 4679-87, (2002)). In some embodiments, the viral core proteins can include, but are not limited to, a viral Gag protein, for example, a retrovirus gag protein [e.g. a HIV Gag viral protein (e.g., HIV-1 NL43 Gag (GenBank serial number AAA44987)), a simian immunodeficiency virus (SW) Gag viral protein (e.g., SIVmac239 Gag (GenBank serial number CAA68379)), or a murine leukemia virus (MuLV) Gag viral protein (e.g., MuLV Gag (GenBank serial number S70394))], a retrovirus matrix protein, a rhabdovirus matrix protein M protein [e.g., a vesicular stomatis virus (VSV) M protein (e.g., VSV Matrix protein (GenBank serial number NP041714))], a filovirus viral core protein (e.g., an Ebola VP40 viral protein (e.g., Ebola virus VP40 (GenBank serial number AAN37506))), a Rift Valley Fever virus N protein (e.g., RVFV N Protein (GenBank serial number NP049344)), a coronavirus M, E and NP protein (e.g., GenBank serial number NP040838 for NP protein, NP 040835 for M protein, CAC39303 for E protein of Avian Infections Bronchitis Virus and NP828854 for E protein of the SARS virus)), a bunyavirus N protein (GenBank serial number AAA47114)), an influenza Ml protein, a paramyxovirus M protein, an arenavirus Z protein (e.g., a Lassa Fever Virus Z protein), and combinations thereof.

In some aspects, the present disclosure provides methods of inducing in a human subject an immune response to HIV, the methods comprising administering to the subject a first lipid nanoparticle comprising a mRNA encoding an HIV envelope (Env) protein from a first Clade, preferentially a UCA-engaging Env, and a mRNA encoding a lentivirus group-specific antigen (Gag) protein, and administering to the subject a second lipid nanoparticle comprising a mRNA encoding an HIV Env protein from a second Clade and a mRNA encoding a lentivirus Gag protein, wherein the first lipid nanoparticle and the second lipid nanoparticle are administered more than once and in an amount effective at inducing in the subject a population of neutralizing antibodies that bind to shared epitopes on proteins from the first Clade and neutralizing antibodies that bind to shared epitopes on proteins from the second Clade. In some embodiments, the HIV is HIV Type 1 (HIV-1).

In some embodiments, the population comprises neutralizing antibodies that bind to shared epitopes on proteins from multiple strains of the first Clade and neutralizing antibodies that bind to shared epitopes on proteins from multiple strains of the second Clade.

In some embodiments, the methods further comprise administering to the subject at least one additional lipid nanoparticle comprising a mRNA encoding an HIV Env protein from at least one additional Clade and a mRNA encoding an HIV Gag protein. Thus, in some embodiments, the population comprises neutralizing antibodies that bind to shared epitopes on proteins from multiple strains of the at least one additional Clade.

In some embodiments, the first nanoparticle comprises a ratio of mRNA encoding an HIV Env protein to mRNA encoding an HIV Gag protein of at least 1:1, and/or wherein the second nanoparticle comprises a ratio of mRNA encoding an HIV Env protein to mRNA encoding an HIV Gag protein of at least 1:1. For example, the first nanoparticle may comprise a ratio of the mRNA encoding an HIV Env protein to the mRNA encoding an HIV Gag protein of at least 3:2, and/or the second nanoparticle may comprise a ratio the mRNA encoding an HIV Env protein to the mRNA encoding an HIV Gag protein of at least 3:2.

In some embodiments, the first nanoparticle comprises a ratio of mRNA encoding an HIV Env protein to mRNA encoding furin of at least 5:1. In some embodiments, the ratio of mRNA encoding an HIV Gag protein to mRNA encoding a viral protease is at least 5:1. In some embodiments, the first nanoparticle comprises mRNA encoding the full or partially truncated Gag-Pol gene from HIV-1 or SIV, which comprises the viral protease gene, thus providing an alternative to an mRNA encoding a viral protease.

In some embodiments, the HIV Env protein comprises mutations, relative to wild-type HIV Env protein, that favor a closed conformation. In some embodiments, the HIV Env protein comprises mutations, relative to wild-type HIV Env protein, that comprises glycan knock-in or knock-out modifications. In some embodiments, the HIV Env protein is a stabilized soluble Env protein. For example, HIV Env protein may be an HIV Env SOSIP.664 protein (e.g., SEQ ID NO: 9). In some embodiments, the HIV Env protein is a membrane-bound HIV Env protein. In some embodiments, the cytosolic portion of the HIV Env protein is partially truncated. For example, the membrane-bound HIV Env protein may be gp150 with a truncation at position 745, or gp160.

In some embodiments, the lentivirus is selected from HIV, simian immunodeficiency virus (SIV), and murine leukemia virus (muLV). In some embodiments, the lentivirus-derived Gag is replaced by VSV-AG or VSV core protein.

In some embodiments, the first Clade, the second Clade, and the at least one additional Clade are selected from the group consisting of HIV Group M Clades A-K and related circulating recombinant forms (CRFs).

In some embodiments, the first lipid nanoparticle and the second nanoparticle are administered sequentially. In other embodiments, the first lipid nanoparticle and the second nanoparticle are administered simultaneously.

In some embodiments, the first lipid nanoparticle is administered as multiple doses separated by at least 1 week per administration, prior to administration of the second lipid nanoparticle. In some embodiments, the second lipid nanoparticle is administered as multiple doses separated by at least 1 week per administration, after administration of the first lipid nanoparticle. In some embodiments, the second lipid nanoparticle and the at least one additional lipid nanoparticle are administered simultaneously.

In some embodiments, a first Clade is Clade A (e.g., Clade A, Clade AC, Clade AE, or Clade AG). For example, an HIV Env protein may be an HIV Clade A BG505, Q23, Q842, MI369, KER2008, 0330, RW020 or B1369 strain Env protein, an HIV Clade AC 3301 strain Env protein, an HIV Clade AE C2101, CM244 or BJOXO28000 strain Env protein, or an HIV Clade AG DJ263 or T280 strain Env protein.

In some embodiments, a first Clade is Clade B. For example, an HIV Env protein may be an HIV Clade B WITO strain Env protein with or without removal of glycans at positions 188, 276, 460 and 463 to better engage UCA antibodies. In some embodiments, the HIV Env protein is selected from HIV Clade B WITO, X2278, JRCSF, JR-FL, B41, 3988, 45_01dG5, BX08, RHPA, TRJO, YU2 or REJO strain Env Proteins.

In some embodiments, a first Clade is Clade C. For example, an HIV Env protein may be an HIV Clade C 426c strain Env protein with or without removal of glycans at positions 188, 276, 460 and 463 to better engage UCA antibodies. In some embodiments, the HIV Env protein is selected from HIV Clade C DU422, 426C, CH505, ZM176, ZM249, ZA012, DU156, CH848, CH1012, MM24, MM45, 001428, BR025, or MW965 strain Env proteins.

In some embodiments, a first Clade is Clade D. For example, an HIV Env protein may be an HIV Clade D A07412M1 strain Env protein.

In some embodiments, a first Clade is Clade G. For example, an HIV Env protein may be an HIV Clade G X1193 or P1981 strain Env protein.

In some embodiments, a second Clade is Clade A (e.g., Clade A, Clade AC, Clade AE, or Clade AG). For example, an HIV Env protein may be an HIV Clade A BG505, Q23, Q842, MI369, KER2008, 0330, RW020 or B1369 strain Env protein, an HIV Clade AC 3301 strain Env protein, an HIV Clade AE C2101, CM244 or BJOXO28000 strain Env protein, or an HIV Clade AG DJ263 or T280 strain Env protein.

In some embodiments, a second Clade is Clade B. For example, an HIV Env protein may be an HIV Clade B WITO strain Env protein with or without removal of glycans at positions 188, 276, 460 and 463 to better engage UCA antibodies. In some embodiments, the HIV Env protein is selected from HIV Clade B WITO, X2278, JRCSF, JR-FL, B41, 3988, 45_01dG5, BX08, RHPA, TRJO, YU2 or REJO strain Env Proteins.

In some embodiments, a second Clade is Clade C. For example, an HIV Env protein may be an HIV Clade C 426c strain Env protein with or without removal of glycans at positions 188, 276, 460 and 463 to better engage UCA antibodies. In some embodiments, the HIV Env protein is selected from HIV Clade C DU422, 426C, CH505, ZM176, ZM249, ZA012, DU156, CH848, CH1012, MM24, MM45, 001428, BR025, or MW965 strain Env proteins.

In some embodiments, a second Clade is Clade D. For example, an HIV Env protein may be an HIV Clade D A07412M1 strain Env protein.

In some embodiments, a second Clade is Clade G. For example, an HIV Env protein may be an HIV Clade G X1193 or P1981 strain Env protein.

In some embodiments, the HIV Env protein comprises a sequence of an Env protein of an HIV strain obtained from an infected subject who has broad and potent neutralizing antibodies to HIV Env protein. In some embodiments, the HIV Env protein comprises a consensus sequence of variants an Env protein of an HIV strain obtained from an infected subject who has broad and potent neutralizing antibodies to HIV Env protein.

In some embodiments, the methods comprise administering to the subject a first lipid nanoparticle comprising a mRNA encoding a first HIV Env protein and a mRNA encoding an HIV Gag polyprotein, administering to the subject a second lipid nanoparticle comprising a mRNA encoding a second HIV Env protein and a mRNA encoding an HIV Gag polyprotein, and administering to the subject a third lipid nanoparticle comprising a mRNA encoding a third HIV Env protein and a mRNA encoding an HIV Gag polyprotein, wherein the population of neutralizing antibodies comprises neutralizing antibodies that bind to shared epitopes on proteins from multiple different HIV strains. In some embodiments, at least one of the first, second, and third HIV Env proteins comprises a sequence of an Env protein of an HIV strain obtained from an infected subject who has broad and potent neutralizing antibodies to HIV Env protein. In some embodiments, at least one of the first, second, and third HIV Env proteins comprises a consensus sequence of variants an Env protein of an HIV strain obtained from an infected subject who has broad and potent neutralizing antibodies to HIV Env protein.

In some embodiments, the methods comprise administering to the subject a first lipid nanoparticle comprising a mRNA encoding an HIV Clade B Env protein and a mRNA encoding an HIV Gag polyprotein, administering to the subject a second lipid nanoparticle comprising a mRNA encoding an HIV Clade A Env protein and a mRNA encoding an HIV Gag polyprotein, and administering to the subject a third lipid nanoparticle comprising a mRNA encoding an HIV Clade C Env protein and a mRNA encoding an HIV Gag polyprotein, wherein the population of neutralizing antibodies comprises neutralizing antibodies that bind to shared epitopes on HIV Clade B proteins, HIV Clade A proteins, and HIV Clade C proteins. In some embodiments, the population of neutralizing antibodies comprises neutralizing antibodies that bind to shared epitopes on at least 3 Clades comprised in the group-M (Clades A-K) HIV viruses.

In some embodiments, the population comprises neutralizing antibodies that bind to shared epitopes on proteins from multiple Clade B HIV strains, neutralizing antibodies that bind to shared epitopes on proteins from multiple Clade A HIV strains, and neutralizing antibodies that bind to shared epitopes on proteins from multiple Clade C HIV strains. In some embodiments, the population comprises neutralizing antibodies that bind to shared epitopes on proteins from at least five (5) different HIV strains. For example, the population may comprise neutralizing antibodies that bind to shared epitopes on proteins from at least 10 different HIV strains. In some embodiments, the population comprises neutralizing antibodies that bind to shared epitopes on proteins from any two or more (e.g., 2, 3, 4, 5, 6, 7, 8, 9, 10, 11, 12, 13, 14, 15, 16, 17, 18, 19, or 20) of the following HIV strains: JRFL, WITO.33, BG505, AD8, 398F1, CNE8, CNE55, 25710, CE1176, X1632, TRO11, X2278, BJOXO2000, X2632, 246F3, CH119, CE0217, A3, 02, and A3/02.

In some embodiments, none of the first, second, or at least one additional lipid nanoparticles comprise mRNA encoding a soluble HIV Env protein.

Also provided herein are immunogenic compositions comprising a lipid nanoparticle comprising mRNA encoding a membrane-bound HIV Env protein and a mRNA encoding an HIV Gag protein, wherein the lipid nanoparticle comprises an ionizable cationic lipid, a non-cationic lipid, sterol, and a PEG-modified lipid. In some embodiments, the immunogenic compositions comprise a lipid nanoparticle comprising mRNA encoding an HIV Env SOSIP.664 protein and a mRNA encoding an HIV Gag protein. In some embodiments, the immunogenic compositions comprise a lipid nanoparticle comprising mRNA encoding a membrane-bound HIV Env protein and a mRNA encoding an HIV Gag protein, wherein the ratio of the mRNA encoding a membrane-bound HIV Env protein to the mRNA encoding an HIV Gag protein is at least 3:2.

### BRIEF DESCRIPTION OF THE DRAWINGS

FIG. 1A shows a schematic of the co-formulation used herein, with mRNA encoding HIV Env and mRNA encoding lentivirus Gag, which enables in vivo production of extracellular virus-like particles (VLPs), mimicking natural infection. FIG. 1B shows a schematic of the HIV-1 maturation process.

FIG. 2 shows the study plan with associated vaccination doses, components, Clades, and timelines.

FIG. 3 shows a graph of an autologous titer of WITO.27 tier 2 (Clade B) of Rhesus macaques. The median inhibitory dose (“ID₅₀”) is plotted on the y-axis against time displayed in weeks on the x-axis. The vaccination doses and respective Clade information are also indicated below the x-axis to indicate the timing and identity of the vaccination doses.

FIG. 4 shows the infection-free survival out to day 56 of dosed Rhesus macaques from Example 1 (contained herein below) after repeated low-dose intra-rectal inoculation of tier-2 heterologous SHIV virus (AD8). The percent survival is represented on the y-axis with the time elapsed in days on the x-axis.

FIG. 5 shows the infection-free survival out to day 56 of dosed Rhesus macaques from Example 1 (contained herein below) after repeated low-dose intra-rectal inoculation of tier-2 heterologous SHIV virus (AD8). The percent survival is represented on the y-axis with the time elapsed in days on the x-axis.

FIG. 6 shows that the SIV Gag polyprotein is efficiently processed to its final products, including the main core protein p27, in the presence of SIV protease.

FIG. 7 shows that fully processed SIV core protein p27 is efficiently and selectively incorporated into virus-like particles (VLPs) in the presence of SIV protease.

FIG. 8 shows that the production of extracellular SHIV VLPs is markedly increased in the presence of SIV protease.

FIGS. 9A-9B show that the efficiency of Env processing to gp120 (FIG. 9A) and the production of extracellular VLPs (FIG. 9B) are markedly increased in the presence of furin.

FIG. 10 shows that both HIV-1 Env and Gag are fully processed in virus-like particles (VLPs) produced using Gag-Pol (which expresses also the viral protease).

FIG. 11 shows that VLPs produced using Gag-Pol display a desired vaccine-relevant antigenic profile, with high expression of epitopes recognized by broadly neutralizing antibodies (bNAbs).

FIG. 12 shows detection and sorting of single B cells producing antibodies to the CD4-binding site from the peripheral blood of a representative macaque that was completely protected from SHIV-AD8 infection. B cells double-positive for labelled BG505 trimer (y-axis) and eODGT8.M49 (x-axis) probes were individually sorted by FACS. Antibody cloning and expression of paired heavy and light chains are in progress.

FIG. 13 shows FACS-Ssorted CD4-BS-specific B cells from which productive antibodies were cloned.

FIG. 14 shows prevalence of heavy chain variable gene (VH) usage among CD4-BS-specific B cells from a protected macaque.

FIG. 15 shows variation from germline sequence in heavy chain variable genes (VH) among CD4-BS-specific B cells.

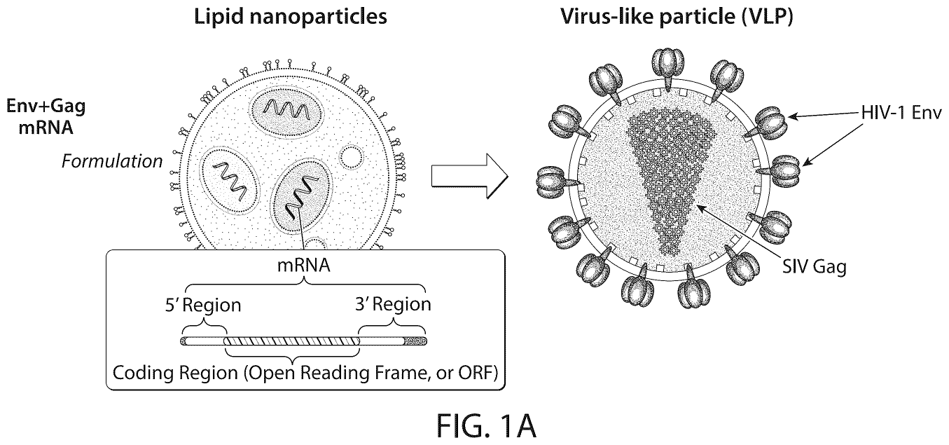

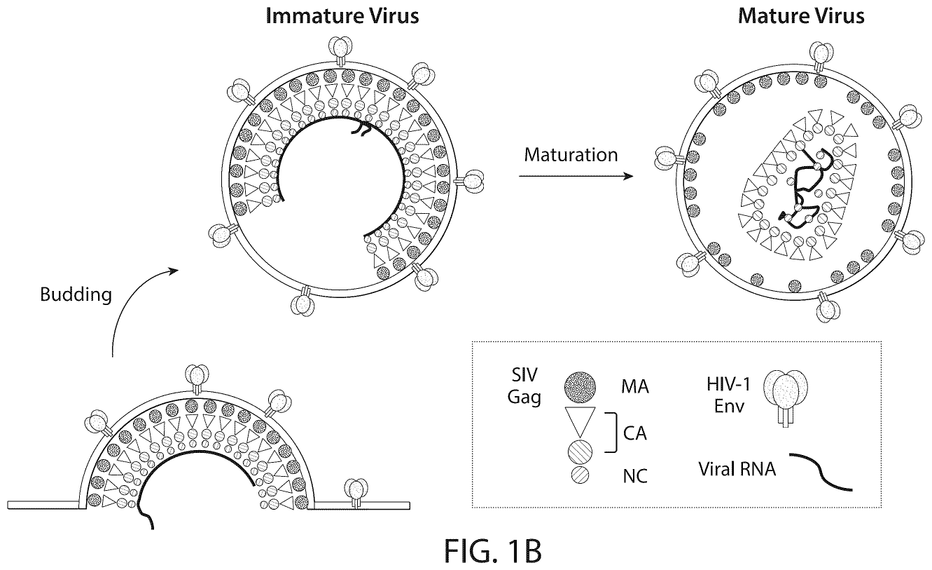

### DETAILED DESCRIPTION

In some aspects, the present disclosure provides methods of inducing in a human subject an immune response to HIV, the methods comprising administering to the subject a first lipid nanoparticle comprising a mRNA encoding an HIV envelope (Env) protein from a first Clade and a mRNA encoding a lentivirus group-specific antigen (Gag) protein, and administering to the subject a second lipid nanoparticle comprising a mRNA encoding an HIV Env protein from a second Clade and a mRNA encoding a lentivirus Gag protein.

### Human Immunodeficiency Virus (HIV)

Some aspects of the disclosure provide methods of inducing in a subject an immune response to human immunodeficiency virus (HIV). HIV (Retroviridae, Orthoretrovirinae) is a lentivirus capable of long-term latent infection of cells and short-term cytopathic effects, which can produce the progressive and fatal acquired immunodeficiency syndrome (AIDS). HIV causes depletion of host T-cells, primarily CD4+ T-cells, thereby leaving the host unable to fight off serious as well as otherwise innocuous infections. An infectious HIV virion includes two identical strands of RNA that are packaged within a core of viral proteins. The virion core is surrounded by a phospholipid bilayer envelope largely derived from the host cell membrane and includes proteins encoded in the viral RNA.

Two major and closely related strains of HIV have been identified, HIV type 1 (HIV-1) and HIV type 2 (HIV-2), of which HIV-1 is the predominate strain and recognized as the more virulent and aggressive form of HIV accounting for approximately 95% of all HIV infections world-wide, with HIV-2 being relatively uncommon and less infectious. In some embodiments, the methods of the disclosure induce an immune response to HIV-1. The present disclosure contemplates HIV vaccines that can equally target and/or be effective in treating or preventing any strain of HIV.

The integrated form of HIV-1, also known as the provirus, is approximately 9.8 kilobases in length. Both ends of the provirus are flanked by a repeated sequence Referred to as the long terminal repeats (LTRs). The genes of HIV are located in the central region of the proviral DNA and encode at least nine proteins, divided into three classes: the major structural proteins, Gag, Pol, and Env: the regulatory proteins, Tat and Rev; and the accessory proteins, Vpu, Vpr, Vif, and Nef. The mRNA vaccines of the present disclosure, in some embodiments, comprise mRNA encoding HIV Env protein and/or lentivirus (e.g., HIV) Gag protein.

### HIV Env

The Env protein forms part of the HIV viral envelope. The env gene encodes glycoprotein 160 (gp160), which is cleaved by the host cell protease yielding glycoprotein 120 (gp120) and glycoprotein 41 (gp41) subunits, with the external gp120 subunit non-covalently bonded to the transmembrane gp41 subunit. This resulting Env complex, which includes three gp120/gp4l pairs, mediates a multistep process that results in the fusion of the viral membrane with the host cell membrane. In the first step of the process, gp120 facilitates initial binding with host cells, most commonly through CD4 receptors, which in turn fosters binding of a chemokine co-receptor. The most common chemokine receptors to act as co-receptors are CXCR4 and CCR5, however additional chemokine receptors, as well as various other proteins of the G protein-coupled receptor superfamily, have been shown to serve as co-receptors for HIV entry. Co-receptor binding causes gp41 to undergo a conformational change exposing a hydrophobic region to the host cell membrane. Gp41 then inserts itself into the host cell causing gp41 to again change conformation and fold back on itself forcing fusion of the host cell membrane with the viral membrane, in turn enabling the viral capsid to enter the cell. Env can genetically vary by as much as 35% across Clades causing variation in the precise genetic sequence of the protein, which causes distinct tropisms. These tropisms are often related to the specificity of the gp120 variants to different chemokine receptors, thus influencing virion binding, altering membrane fusion triggers, and ultimately the mechanism of host cell infection. Env protein is also assembled post viral DNA integration into the host genome. Once transcribed, the viral RNA is transported to the cytoplasm, where Env protein is generated. Post translation, the protein is integrated into the host cell membrane, a process facilitated by Gag polyprotein.

The HIV Env proteins of the present disclosure may be one of several different HIV Clades. HIV-1, for example, is divided into four known groups (M, N, O, and P), which are believed to represent an independent transmission of simian immunodeficiency virus (SIV) into humans. Of the four groups, M is considered the major group (accounting for more than 90% of HIV and AIDS cases by some estimates), with N, O, and P representing the minor groups. Within the M group of HIV-1, eleven sub-types, referred to as Clades A-K, have been established, which unlike the groups do not indicate distinct transmissions of SIV, but rather often refer to the geographic area the Clade is predominate. Similar to the four groups, Clades have been assigned letter designations A-K, but more may be established. Further variations of the known Clades can result from genetic combinations of the Clades, resulting in hybrids known as circulating recombinant forms (CRF) (e.g., A/B, C/K, or any combination of A-K).

The HIV Env proteins encoded by the mRNA administered herein may be, and in preferred embodiments are, from different Clades, for example, one from a “first Clade” (e.g., A-K) and one from a second Clade (e.g., any one of A-K that is not the first Clade) (and optionally at least one additional Clade, e.g., any one of A-K that is not the first or second Clade. In some embodiments, the first Clade, the second Clade, and the at least one additional Clade are selected from the group consisting of HIV Group M Clades A-K and related circulating recombinant forms (CRFs). The first, the second Clade, and the at least one additional Clade may be selected, in some embodiments, from Clade A of Group M, Clade AC of Group M, Clade AE of Group M, Clade AG of Group M. Clade B of Group M, Clade C of Group M, Clade D of Group M, Clade E of Group M, Clade F of Group M, Clade G of Group M, Clade H of Group M, Clade I of Group M, Clade J of Group M, or Clade K of Group M. In some embodiments, the first Clade is Clade A of Group M. In some embodiments, the first Clade is Clade AC of Group M. In some embodiments, the first Clade is Clade AE of Group M. In some embodiments, the first Clade is Clade AG of Group M. In some embodiments, the first Clade is Clade B of Group M. In some embodiments, the first Clade is Clade C of Group M. In some embodiments, the first Clade is Clade B of Group M. and the second Clade is Clade A of Group M. In some embodiments, the first Clade is Clade B of Group M, and the second Clade is Clade C of Group M.

In some embodiments, the first Clade is Clade B. For example, the HIV Env protein may be an HIV Clade B WITO, X2278, JRCSF, JR-FL, B41, 3988, 45_01dG5, BX08, RHPA, TRJO, YU2, or REJO strain Env protein. In some embodiments, the second Clade is Clade A, for example, an HIV Clade A BG505, Q23, Q842, MI369, KER2008, 0330, RW020, or B1369 strain Env protein. In some embodiments, the second Clade is Clade C, for example, an HIV Clade C DU422, 426C, CH505, ZM176, ZM249, ZA012, DU156, CH848, CH1012, MM24, MM45, 001428, BR025, or MW965strain Env protein.

The Env proteins encoded by the HIV mRNA vaccines of the present disclosure induce protective titers of broadly neutralizing antibodies against multiple strains of HIV (e.g., multiple strains of HIV-1). These neutralizing antibodies bind to the Env complex on the virion surface to neutralize HIV infectivity. Unlike more common strategies for broad neutralizing antibody induction that involve the design of soluble, recombinant protein mimics of the native Env complex, the HIV mRNA vaccines provided herein, in some embodiments, encode membrane-bound Env protein, such as gp160, or a truncated form, gp150 (cytosolic portion truncated at residue 775, relative to the reference HIV Env protein). Herein, the “reference HIV Env protein” is a soluble, stabilized trimeric HIV Env SOSOP.644 complex; Sanders R W et al. PLoS Pathog 9:e1003618, incorporated herein by reference). This soluble reference HIV Env protein includes an engineered disulfide bond that covalently links subunits gp120 and gp41_{ECTO} (produced by introducing a stop codon to truncate the gp41 ectodomain) and also includes an Ile-to-Pro change at residue 559 (I559P) that helps maintain the gp41_{ECTO} moieties in the prefusion form (Binley J M et al. J Virol 2000; 74: 627-643; and Sanders R W et al. J Virol 2002; 76: 8875-8889, each of which is incorporated herein by reference) as well as a Ala-to-Cys change at residue 501 (A501C) and a Thr-to-Cys at residue 605 (T605C). In addition, the truncation of gp41_{ECTO} at residue 664 eliminates a hydrophobic region that tends to cause trimer aggregation (Kliayat R et al. J Virol 2013; 87:9873-9885; Wu X et al. J Virol 2006; 80: 835-844, each of which is incorporated herein by reference).

### Stabilized Trimeric HIV Env SOSOP.644 Complex

MDANKRGLCCVLLLCGAVFVSPSQEIHARFRRGARAENLWVTVYYG
VPVWKDAETTLFCASDAKAYETEKENVWATHACVPTDPNPQEIHL
ENVTEEFNMWKNNMVEQMHTDIISLWDQSLKPCVKLTPLCVTLQC
TNVTNNITDDMRGELKNCSFNMTTELRDKKQKVYSLFYRLDVVQI
NENQGNRSNNSNKEYRLINCNTSAITQACPKVSFEPIPIHYCAPA
GFAILKCKDKKFNGTGPCPSVSTVQCTHGIKPVVSTQLLLNGSLA
EEEVMIRSENITNNAKNILVQFNTPVQINCTRPNNNTRKSIRIGP
GQAFYATGDIIGDIRQAHCNVSKATWNETLGKVVKQLRKHFGNNT
IIRFANSSGGDLEVTTHSFNCGGEFFYCNTSGLFNSTWISNTSVQ
GSNSTGSNDSITLPCRIKQIINMWQRIGQAMYAPPIQGVIRCVSN
ITGLILTRDGGSTNSTTETFRPGGGDMRDNWRSELYKYKVVKIEP
LGVAPTRCKRRVVGRRRRRRAVGIGAVFLGFLGAAGSTMGAASMT
LTVQARNLLSGIVQQQSNLRRAREAQQHLLKLTVWGIKQLQARVL
AVERYLRDQQLLGIWGCSGKLICCTNVPWNSSWSNRNLSEIWDNM
TWLQWDKEISNYTQIIYGLLEESQNQQEKNEQDLLALD*

Several variant forms of HIV Env protein may be encoded by the mRNA of the vaccines provided herein.

The HIV Env proteins, for example, may include one or more CD4 primate binding modifications. These modifications include any of the mutations selected from 153E and 375Y, relative to a reference HIV Env protein comprising the sequence of SEQ ID NO: 1. In some embodiments, a mRNA encodes an HIV Env protein comprising a 153E mutation, relative to a reference HIV Env protein comprising the sequence of SEQ ID NO:1. In some embodiments, a mRNA encodes an HIV Env protein comprising a 375Y mutation, relative to a reference HIV Env protein comprising the sequence of SEQ ID NO:1. Thus, in some embodiments, the membrane-bound form of HIV Env protein comprises mutations, relative to the reference HIV Env protein, that favor a closed conformation, and thus, cannot bind CD4.1

```
(SEQ ID NO: 1)
    1 mrvkekyqhl wrwgwrwgtm llgmlmicsa teklwvtvyy gvpvwkeatt tlfcasdaka
  61 ydtevhnvwa thacvptdpn pqevvlvnvt enfnmwkndm veqmhediis lwdqslkpcv
121 kltplcvslk ctdlkndtnt nsssgrmime kgeikncsfn istsirgkvq keyaffykld
181 iipidndtts ykltscntsv itqacpkvsf epipihycap agfailkcnn ktfngtgpct
241 nvstvqcthg irpvvstqll lngslaeeev virsvnftdn aktiivqlnt sveinctrpn
301 nntrkririq rgpgrafvti gkignmrqah chisrakwnn tlkqiasklr eqfgnnktii
361 fkqssggdpe ivthsfncgg effycnstql fnstwfnstw stegsnnteg sdtitlpcri
421 kgiinmwqky gkamyappis gqircssnit gllltrdggn snneseifrp gggdmrdnwr
481 selykykvvk ieplgvaptk akrrvvqrek ravgigalfl gflgaagstm gaasmtitvq
541 arglisgivq qqnnilraie aqqhllqltv wgikqlqari laverylkdq qllgiwgcsg
601 klicttavpw naswsnksle qiwnhttwme wdreinnyts lihslieesq nqqeknegel
661 leldkwaslw nwfnitnwlw yiklfimivg glvglrivfa visivnrvrg gysplsfqth
721 lptprgpdrp egieeegger drdrsirlvn gslaliwddl rslclfsyhr lrdlllivtr
781 ivellgrrgw ealkywwnll gywsqelkns avsllnatai avaegtdrvi evvqgacrai
841 rhiprrirqg lerill
```

HIV Env proteins having glycan post-translational modifications are also contemplated herein. These modifications may be made at a residue selected from, for example, residue 276 (276N), 295 (295N), 322 (322N), and 386 (386), relative to a reference HIV Env protein comprising the sequence of SEQ ID NO:1. In some embodiments, a mRNA encodes an HIV Env protein comprising a glycan modification at residue 276 (276N), relative to a reference HIV Env protein comprising the sequence of SEQ ID NO:1. In some embodiments, a mRNA encodes an HIV Env protein comprising a glycan modification at residue 295 (295N), relative to a reference HIV Env protein comprising the sequence of SEQ ID NO:1. In some embodiments, a mRNA encodes an HIV Env protein comprising a glycan modification at residue 322 (322N), relative to a reference HIV Env protein comprising the sequence of SEQ ID NO:1. In some embodiments, a mRNA encodes an HIV Env protein comprising a glycan modification at residue 386 (386N), relative to a reference HIV Env protein comprising the sequence of SEQ ID NO:1.

In some embodiments, the HIV Env protein comprises mutations, relative to wild-type HIV Env protein, that comprises glycan knock-in or knock-out modifications. In some embodiments, the HIV Env protein comprises glycan knock-in mutations at residues selected from N332, N241, and N289, relative to an HIV BG550 strain Env protein. In some embodiments, the HIV Env protein comprises glycan knock-in mutations at residues selected from N295 and N386, relative to an HIV DU422, DU172.17, ZM176.66, CNE58, or 426c strain Env protein. In some embodiments, the HIV Env protein comprises glycan knock-in mutations at residue N188, relative to an HIV WITO strain Env protein. In some embodiments, the HIV Env protein comprises glycan knock-out mutations at residue N276, relative to an HIV BG550 strain Env protein.

In some embodiments, the HIV isolate/strain is WITO4160.27. In some embodiments, an HIV mRNA encode membrane-bound Env gp150 (truncated at residue 775) that includes a 153E mutation and a 276D, relative to a reference HIV Env protein, wherein the reference HIV Env protein comprises the sequence of SEQ ID NO: 2. In some embodiments, an HIV mRNA encode membrane-bound Env gp150 (truncated at residue 775) that includes a 153E mutation, a 276D mutation, and a 113C-432GCG disulfide bridge, relative to a reference HIV Env protein, wherein the reference HIV Env protein comprises the sequence of SEQ ID NO: 2.

```
(SEQ ID NO: 2; GenBank: ABG67917.1; WITO4160.27)
    1 mkvmgtkkny qhlwrwgiml lgmlmmssaa eqlwvtvyyg vpvwreantt lfcasdakay
  61 dtevhnvwat hacvptdpnp qevvmgnvte dfnmwknnmy eqmhediisl wdqslkpcvk
121 ltplcvtlhc tnvtisstng stanvtmreg mkncsfnttt virdkiqkey alfykldivp
181 iegkntntsy rlincntsvi tqacpkvsfe pipihycapa gfailkcnnk tingkgpcrn
241 vstvqcthgi kpvvstqlli ngslaeedii irsenftnng kniivqlkep vkinctrpgn
301 ntrrsinigp grafyatgai igdirkahcn isteqwnntl tqivdklreq fgnktiifnq
361 ssggdpevvm htfncggeff ycnstalfns twfnngtstw nstadnitlp crikqvinmw
421 qevgkamyap pirgqidcss nitgliltrd ggsnssqnet frpgggnmkd nwrselykyk
481 vvkieplgia ptrakrrvvq rekravtlga vflgflgaag stmgaasltl tvqarlllsg
541 ivqqqsnllr aieaqqhmlq ltvwgikqlq arvlaieryl kdqqllgiwg csgklicttt
601 vpwntswsnk sydyiwnnmt wmqwereidn ytgfiytlie esqnqqekne lelleldkwa
661 slwnwfnitn wlwyiklfim iigglvglrt vcavlsivnr vrcqgsplsf qtrlpnprgp
721 grpeetegeg gerdrdrsar lvngflaiiw ddlrslclfs yhrlrdllli varvveilgr
781 rgweilkyww nllkywsqel knsavsllnv taiavaegtd rvieivqrav railhiptri
841 rqgferall
```

In some embodiments, the HIV isolate/strain is DU422, DU172.17, ZM176.66, CNE58, or 426c. In some embodiments, an HIV mRNA encode membrane-bound Env gp150 (truncated at residue 775) that includes a 295N mutation, a 386N mutation, and a 375Y mutation, relative to a reference HIV Env protein, wherein the reference HIV Env protein comprises the sequence of SEQ ID NO: 3. In some embodiments, an HIV mRNA encode membrane-bound Env gp150 (truncated at residue 775) that includes a 295N mutation, a 386N mutation, a 375Y mutation, and a 133C-432GCG disulfide bridge, relative to a reference HIV Env protein, wherein the reference HIV Env protein comprises the sequence of SEQ ID NO: 3.

```
(SEQ ID NO: 3; GenBank: ABD83641.1; Du422.1)
    1 mrvrgiprnw pqwwiwgilg fwmiiicrvv gnldlwvtvy ygvpvwkeak ttlfcasdak
  61 aydkevhnvw athacvptdp npqeivlenv tenfnmwknd mvdgmhedii slwdqslkpc
121 vkltplcvtl ncknvnisan anatatlnss mngeikncsf ntttelrdkk qkvyalfykp
181 dvvplnggeh netgeyilin cnsstitqac pkvsfdpipi hycapagyai lkcnnktfng
241 tgpcnnvstv qcthgikpvv stqlllngsl aeeeiivrse nltnniktii vhinksveik
301 ctrpnnntrk svrigpgqtf yatgeligdi reahcnisre twistliqvk eklrehynkt
361 ikfepssggd levtthsinc rgeffycdtt klfnetklin eseyvdnkti ilpcrikqii
421 nmwgevgram yappiegnit cksnitgill twdggenste gvfrpgggnm kdnwrselyk
481 ykvveikplg vaptkskrkv vgrekravgl gavllgflga agstmgaasi tltvqarqll
541 sgivqqqsnl lraieaqqhl lqltvwgikq lqtrvlaier yikdqqllgl wgcsgklica
601 tavpwnssws nkslgdiwan mtwmqwdrei snytntifrl ledsqnqgek nekdllalds
661 wknlwnwfdi tnwlwyikif imivggligl riifgvlaiv krvrqgyspl sfqtlipnpr
721 gpdrlgriee eggeqdkdrs irlvsgflal awddlrslcl fsyhqlrdfi ltaaraaell
781 grsslrglqr gwevlkylgn lvqywglelk rsainlfdti aiavaegtdr iievigricr
841 airyiptrir ggfeaall
```

In some embodiments, the HIV isolate/strain is BG505. In some embodiments, an HIV mRNA encode membrane-bound Env gp160 that includes a 322N mutation and a 375Y mutation, relative to a reference HIV Env protein, wherein the reference HIV Env protein comprises the sequence of SEQ ID NO: 4. In some embodiments, the HIV isolate/strain is BG505. In some embodiments, an HIV mRNA encode membrane-bound Env gp160 that includes a 322N mutation, a 375Y mutation, and a 113C-429GCG disulfide bridge, relative to a reference HIV Env protein, wherein the reference HIV Env protein comprises the sequence of SEQ ID NO: 4.

```
(SEQ ID NO: 4; GenBank: ABA61516.1; BG505)
    1 mrvmgiqrnc qhlfrwgtmi igmiiicsaa enlwvtvyyg vpvwkdaett lfcasdakay
  61 etekhnvwat hacvptdpnp geihlenvte efnmwknnmv eqmhtdiisl wdqslkpcvk
121 ltplcvtlqc tnvtnnitdd mrgelkncsf nmttelrdkk qkvyslfyrl dvvqinenqg
181 nrsnnsnkey rlincntsai tqacpkvsfe pipihycapa gfailkckdk kfngtgpcps
241 vstvqcthgi kpvvstqlll ngslaeeevm irsenitnna knilvqfntp vqinctrpnn
301 ntrksirigp gqafyatgdi igdirgahct vskatwnetl gkvvkqlrkh fgnntiirfa
361 nssggdlevt thsfncggef fycntsglfn stwisntsvq gsnstgsnds itlpcrikqi
421 inmwqrigga myappiqgvi rcvsnitgli ltrdggstns ttetfrpggg dmrdnwrsel
481 ykykvvkiep lgvaptrakr rvvgrekrav gigavflgfl gaagstmgaa smtltvqarn
541 llsgivqqqs nllraieaqq hllkltvwgi kqlqarvlav erylrdqqll giwgcsgkli
601 cttnvpwnss wsnrnlseiw dnmtwlqwdk eisnytqiiy glleesqnqq ekneqdllal
661 dkwaslwnwf disnwlwyik ifimivcgli glrivfavls vihrvrqgys plsfqthtpn
721 prgldrperi eeedgeqdrg rstrlvsgfl alawddlrsl clfcyhrlrd filiaarive
781 llghsslkgl rlgweglkyl wnllaywgre lkisainlfd tiaiavaewt drvieigqrl
841 craflhiprr irgglerall
```

The HIV Env protein, in some embodiments, comprises a sequence of an Env protein of an HIV strain obtained from an infected subject who has broad and potent neutralizing antibodies to HIV Env protein. A subject is considered to have been infected with HIV if, for example, the subject test positive following an HIV viral load test (also referred to as an HIV nucleic acid amplification test (NAAT or NAT); HIV by PCR; or HIV RNA test). Methods for diagnosing an HIV positive subject are known, any of which may be used herein.

In some embodiments, the HIV Env protein comprises a consensus sequence of variants an Env protein of an HIV strain obtained from an infected subject who has broad and potent neutralizing antibodies to HIV Env protein. The consensus sequence may be determined, for example, by aligning the amino acid sequences (or nucleic acid sequences) of various HIV Env proteins obtained from an infected subject who has broad and potent neutralizing antibodies to HIV Env protein, then determining the most commonly expressed amino acid (or nucleic acid) at each position.

### HIV Gag

The lentivirus group-specific antigen (gag) gene encodes a 55-kilodalton (kD) Gag precursor protein, also called p55, which is expressed from the unspliced viral mRNA. During translation, the N terminus of p55 is myristoylated, triggering its association with the cytoplasmic aspect of cell membranes. The membrane-associated Gag polyprotein recruits two copies of the viral genomic RNA along with other viral and cellular proteins that triggers the budding of the viral particle from the surface of an infected cell. After budding, p55 is cleaved by the virally encoded protease (a product of the pol gene) during the process of viral maturation into four smaller proteins designated MA (matrix [p17]), CA (capsid [p24]), NC (nucleocapsid [p9]), and p6 (Göttlinger H G et al. Proc Natl Acad Sci USA 1989; 86(15): 5781-5).

The lentiviral Gag protein encoded herein may be an HIV Gag protein, a simian immunodeficiency virus (SIV) Gag protein, or a murine leukemia virus (muLV) Gag protein. In some embodiments, the Gag protein is an HIV Gag protein. In some embodiments, the Gag protein is a SIV Gag protein. In some embodiments, the Gag protein is an muLV Gag protein. In some embodiments, the SIV Gag protein is a SIVmac239 Gag protein.

```
>HIV_gag (HXB2 genbank AAB50258.1)
(SEQ ID NO: 5)
    1 mgarasvlsg geldrwekir lrpggkkkyk lkhivwasre lerfavnpgl letsegerqi
  61 1gqlqpslqt gseelrslyn tvatlycvhq rieikdtkea ldkieeeqnk skkkaqqaaa
121 dtghsnqvsq nypivqniqg qmvhqaispr tlnawvkvve ekafspevip mfsalsegat
181 pqdlntmlnt vgghqaamqm lketineeaa ewdrvhpvha gpiapgqmre prgsdiagtt
241 stlqeqigwm tnnppipvge iykrwiilgl nkivrmyspt sildirqgpk epfrdyvdrf
301 yktlraegas gevknwmtet llvqnanpdc ktilkalgpa atleemmtac qgvggpghka
361 rvlaeamsqv tnsatimmqr gnfrnqrkiv kcfncgkegh tarncraprk kgcwkcgkeg
421 hqmkdcterq anflgkiwps ykgrpgnflq srpeptappe esfrsgvett tppqkgepid
481 kelypltslr slfgndpssq
```

### HIV Gag-Pol

>HIV_gag_pol (psPAX2)
(SEQ ID NO: 6)
ATGGGTGCGAGAGCGTCAGTATTAAGCGGGGGAGAATTAGATCGATGGGAAAAAATTCGGTTAAGGCCAGGGGGA
AAGAAAAAATATAAATTAAAACATATAGTATGGGCAAGCAGGGAGCTAGAACGATTCGCAGTTAATCCTGGCCTG
TTAGAAACATCAGAAGGCTGTAGACAAATACTGGGACAGCTACAACCATCCCTTCAGACAGGATCAGAAGAACTT
AGATCATTATATAATACAGTAGCAACCCTCTATTGTGTGCATCAAAGGATAGAGATAAAAGACACCAAGGAAGCT
TTAGACAAGATAGAGGAAGAGCAAAACAAAAGTAAGAAAAAAGCACAGCAAGCAGCAGCTGACACAGGACACAGC
AATCAGGTCAGCCAAAATTACCCTATAGTGCAGAACATCCAGGGGCAAATGGTACATCAGGCCATATCACCTAGA
ACTTTAAATGCATGGGTAAAAGTAGTAGAAGAGAAGGCTTTCAGCCCAGAAGTGATACCCATGTTTTCAGCATTA
TCAGAAGGAGCCACCCCACAAGATTTAAACACCATGCTAAACACAGTGGGGGGACATCAAGCAGCCATGCAAATG
TTAAAAGAGACCATCAATGAGGAAGCTGCAGAATGGGATAGAGTGCATCCAGTGCATGCAGGGCCTATTGCACCA
GGCCAGATGAGAGAACCAAGGGGAAGTGACATAGCAGGAACTACTAGTACCCTTCAGGAACAAATAGGATGGATG
ACACATAATCCACCTATCCCAGTAGGAGAAATCTATAAAAGATGGATAATCCTGGGATTAAATAAAATAGTAAGA
ATGTATAGCCCTACCAGCATTCTGGACATAAGACAAGGACCAAAGGAACCCTTTAGAGACTATGTAGACCGATTC
TATAAAACTCTAAGAGCCGAGCAAGCTTCACAAGAGGTAAAAAATTGGATGACAGAAACCTTGTTGGTCCAAAAT
GCGAACCCAGATTGTAAGACTATTTTAAAAGCATTGGGACCAGGAGCGACACTAGAAGAAATGATGACAGCATGT
CAGGGAGTGGGGGGACCCGGCCATAAAGCAAGAGTTTTGGCTGAAGCAATGAGCCAAGTAACAAATCCAGCTACC
ATAATGATACAGAAAGGCAATTTTAGGAACCAAAGAAAGACTGTTAAGTGTTTCAATTGTGGCAAAGAAGGGCAC
ATAGCCAAAAATTGCAGGGCCCCTAGGAAAAAGGGCTGTTGGAAATGTGGAAAGGAAGGACACCAAATGAAAGAT
TGTACTGAGAGACAGGCTAATTTTTTAGGGAAGATCTGGCCTTCCCACAAGGGAAGGCCAGGGAATTTTCTTCAG
AGCAGACCAGAGCCAACAGCCCCACCAGAAGAGAGCTTCAGGTTTGGGGAAGAGACAACAACTCCCTCTCAGAAG
CAGGAGCCGATAGACAAGGAACTGTATCCTTTAGCTTCCCTCAGATCACTCTTTGGCAGCGACCCCTCGTCACAA
TAAAGATAGGGGGGCAATTAAAGGAAGCTCTATTAGATACAGGAGCAGATGATACAGTATTAGAAGAAATGAATT
TGCCAGGAAGATGGAAACCAAAAATGATAGGGGGAATTGGAGGTTTTATCAAAGTAAGACAGTATGATCAGATAC
TCATAGAAATCTGCGGACATAAAGCTATAGGTACAGTATTAGTAGGACCTACACCTGTCAACATAATTGGAAGAA
ATCTGTTGACTCAGATTGGCTGCACTTTAAATTTTCCCATTAGTCCTATTGAGACTGTACCAGTAAAATTAAAGC
CAGGAATGGATGGCCCAAAAGTTAAACAATGGCCATTGACAGAAGAAAAAATAAAAGCATTAGTAGAAATTTGTA
CAGAAATGGAAAAGGAAGGAAAAATTTCAAAAATTGGGCCTGAAAATCCATACAATACTCCAGTATTTGCCATAA
AGAAAAAAGACAGTACTAAATGGAGAAAATTAGTAGATTTCAGAGAACTTAATAAGAGAACTCAAGATTTCTGGG
AAGTTCAATTAGGAATACCACATCCTGCAGGGTTAAAACAGAAAAAATCAGTAACAGTACTGGATGTGGGCGATG
CATATTTTTCAGTTCCCTTAGATAAAGACTTCAGGAAGTATACTGCATTTACCATACCTAGTATAAACAATGAGA
CACCAGGGATTAGATATCAGTACAATGTGCTTCCACAGGGATGGAAAGGATCACCAGCAATATTCCAGTGTAGCA
TGACAAAAATCTTAGAGCCTTTTAGAAAACAAAATCCAGACATAGTCATCTATCAATACATGGATGATTTGTATG
TAGGATCTGACTTAGAAATAGGGCAGCATAGAACAAAAATAGAGGAACTGAGACAACATCTGTTGAGGTGGGGAT
TTACCACACCAGACAAAAAACATCAGAAAGAACCTCCATTCCTTTGGATGGGTTATGAACTCCATCCTGATAAAT
GGACAGTACAGCCTATAGTGCTGCCAGAAAAGGACAGCTGGACTGTCAATGACATACAGAAATTAGTGGGAAAAT
TGAATTGGGCAAGTCAGATTTATGCAGGGATTAAAGTAAGGCAATTATGTAAACTTCTTAGGGGAACCAAAGCAC
TAACAGAAGTAGTACCACTAACAGAAGAAGCAGAGCTAGAACTGGCAGAAAACAGGGAGATTCTAAAAGAACCGG
TACATGGAGTGTATTATGACCCATCAAAAGACTTAATAGCAGAAATACAGAAGCAGGGGCAAGGCCAATGGACAT
ATCAAATTTATCAAGAGCCATTTAAAAATCTGAAAACAGGAAAGTATGCAAGAATGAAGGGTGCCCACACTAATG
ATGTGAAACAATTAACAGAGGCAGTACAAAAAATAGCCACAGAAAGCATAGTAATATGGGGAAAGACTCCTAAAT
TTAAATTACCCATACAAAAGGAAACATGGGAAGCATGGTGGACAGAGTATTGGCAAGCCACCTGGATTCCTGAGT
GGGAGTTTGTCAATACCCCTCCCTTAGTGAAGTTATGGTACCAGTTAGAGAAAGAACCCATAATAGGAGCAGAAA
CTTTCTATGTAGATGGGGCAGCCAATAGGGAAACTAAATTAGGAAAAGCAGGATATGTAACTGACAGAGGAAGAC
AAAAAGTTGTCCCCCTAACGGACACAACAAATCAGAAGACTGAGTTACAAGCAATTCATCTAGCTTTGCAGGATT
CGGGATTAGAAGTAAACATAGTGACAGACTCACAATATGCATTGGGAATCATTCAAGCACAACCAGATAAGAGTG
AATCAGAGTTAGTCAGTCAAATAATAGAGCAGTTAATAAAAAAGGAAAAAGTCTACCTGGCATGGGTACCAGCAC
ACAAAGGAATTGGAGGAAATGAACAAGTAGATAAATTGGTCAGTGCTGGAATCAGGAAAGTACTATTTTTAGATG
GAATAGATAAGGCCCAAGAAGAACATGAGAAATATCACAGTAATTGGAGAGCAATGGCTAGTGATTTTAACCTAC
CACCTGTAGTAGCAAAAGAAATAGTAGCCAGCTGTGATAAATGTCAGCTAAAAGGGGAAGCCATGCATGGACAAG
TAGACTGTAGCCCAGGAATATGGCAGCTAGATTGTACACATTTAGAAGGAAAAGTTATCTTGGTAGCAGTTCATG
TAGCCAGTGGATATATAGAAGCAGAAGTAATTCCAGCAGAGACAGGGCAAGAAACAGCATACTTCCTCTTAAAAT
TAGCAGGAAGATGGCCAGTAAAAACAGTACATACAGACAATGGCAGCAATTTCACCAGTACTACAGTTAAGGCCG
CCTGTTGGTGGGGGGGGATCAAGCAGGAATTTGGCATTCCCTACAATCCCCAAAGTCAAGGAGTAATAGAATCTA
TGAATAAAGAATTAAAGAAAATTATAGGACAGGTAAGAGATCAGGCTGAACATCTTAAGACAGCAGTACAAATGG
CAGTATTCATCCACAATTTTAAAAGAAAAGGGGGGATTGGGGGGTACAGTGCAGGGGAAAGAATAGTAGACATAA
TAGCAACAGACATACAAACTAAAGAATTACAAAAACAAATTACAAAAATTCAAAATTTTCGGGTTTATTACAGGG
ACAGCAGAGATCCAGTTTGGAAAGGACCAGCAAAGCTCCTCTGGAAAGGTGAAGGGGCAGTAGTAATACAAGATA
ATAGTGACATAAAAGTAGTGCCAAGAAGAAAAGCAAAGATCATCAGGGATTATGGAAAACAGATGGCAGGTGATG
ATTGTGTGGCAAGTAGACAGGATGAGGATTAA

### HIV Protease

HIV-1 protease is a retroviral aspartyl protease essential for the life-cycle of HIV-1 because it cleaves newly synthesized polyproteins at nine cleavage sites to create the mature protein components of an HIV virion. Without effective HIV protease, HIV virions remain uninfectious.

### HIV_Protease GenBank: AEU00248.1

1 pqitlwkrpl vtikiggglk ealldtgadd tvleemnlpg rwkpkmiggi ggfikvrgyd 61 qilieicghk aigtvlvgpt pvniigrnll tqigctlnf (SEQ ID NO: 7)

### Furin

Furin is a host cell enzyme that belongs to the subtilisin-like proprotein convertase family and is responsible for the proteolytic cleavage of the HIV envelope polyprotein precursor gp1160 to gp120 and gp41.

>sp|P09958|FURIN_HUMAN Furin OS = Homo sapiens
OX = 9606 GN = FURIN PE = 1 SV = 2
(SEQ ID NO: 8)
MELRPWLLWVVAATGTLVLLAADAQGQKVFINTWAVRIPGGPAVAN
SVARKHGFLNLGQIFGDYYHFWHRGVTKRSLSPHRPRHSRLQREP
QVQWLEQQVAKRRIKRDVYQEPTDPKEPQQWYLSGVTQRDLNVKA
AWAQGYTGHGIVVSILDDGIEKNHPDLAGNYDPGASFDVNDQDPD
PQPRYTQMNDNRHGTRCAGEVAAVANNGVCGVGVAYNARIGGVRM
LDGEVTDAVEARSLGLNPNHIHIYSASWGPEDDGKTVDGPARLAR
EAFFRGVSQGRGGLGSIFVWASGNGGREHDSCNCDGYINSIYTLS
ISSATQFGNVPWYSEACSSTLATTYSSGNQNEKQIVTTDLRQKCT
ESHTGTSASAPLAAGIIALTLEANKNLTWRDMQHLVVQTSKPAHL
NANDWATNGVGRKVSHSYGYGLLDAGAMVALAQNWTTVAPQRKCI
IDILTEPKDIGKRLEVRKTVTACLGEPNHITRLEHAQARLILSYN
RRGDLAIHLVSPMGIRSTLLAARPHDYSADGENDWAFMTTHSWDE
DPSGEWVLEIENTSEANNYGTLTKFTLVLYGTAPEGLPVPPESSG
CKTLISSQACVVCEEGFSLHQKSCVQHCPPGFAPQVLDTHYSTEN
DVETIRASVCAPCHASCATCQGPALTDCLSCPSHASLDPVEQTCS
RQSQSSRESPPQQQPPRLPPEVEAGQRLRAGLLPSHLPEVVAGLS
CAFIVLVFVIVELVLQERSGFSFRGVKVYTMDRGLISYKGLPPEA
WQEECPSDSEEDEGRGERTAFIKDQSAL
GeneBank: P09958.2

### HIV Immunogenic Compositions/Vaccine Formulations

Provided herein, in some embodiments, are immunogenic compositions comprising a lipid nanoparticle comprising mRNA encoding a membrane-bound HIV Env protein and a mRNA encoding an HIV Gag protein, wherein the lipid nanoparticle comprises an ionizable cationic lipid, a non-cationic lipid, sterol, and a PEG-modified lipid. In some embodiments, the immunogenic compositions comprise a lipid nanoparticle comprising mRNA encoding an HIV Env SOSIP.664 protein (e.g., any of the HIV Env variants described herein) and a mRNA encoding a lentivirus Gag protein.

It should be understood that the HIV mRNA vaccine therapies provided herein include the administration of multiple doses of an HIV mRNA vaccine formulation, each dose separated by at least 1 week, and each dose comprising a combination of mRNA encoding HIV Env protein and mRNA encoding lentivirus Gag protein formulated, for example, in a cationic lipid nanoparticle. It should also be understood that each dose may be different (heterologous) in that the particular HIV strain/isolate from which the mRNA sequence encoding Env protein is obtained/derived may differ and/or the particular dose amount may differ and/or the ratio of mRNA encoding Env v. Gag may differ. Thus, the present disclosure contemplates multiple heterologous boosts of co-formulated mRNA encoding HIV Env and mRNA encoding lentivirus Gag.

In some embodiments, the immunogenic compositions comprise a lipid nanoparticle comprising mRNA encoding a membrane-bound HIV Env protein and a mRNA encoding a lentivirus Gag protein, wherein the ratio of the mRNA encoding a membrane-bound HIV Env protein to the mRNA encoding a lentivirus Gag protein is at least 1:1. For example, the ratio of the mRNA encoding a membrane-bound HIV Env protein to the mRNA encoding a lentivirus Gag protein may be 1:1, 2:1, 3:1, 4:1, or 5:1. In some embodiments, the ratio of the mRNA encoding a membrane-bound HIV Env protein to the mRNA encoding a lentivirus Gag protein is at least 3:2. For example, the ratio of the mRNA encoding a membrane-bound HIV Env protein to the mRNA encoding a lentivirus Gag protein may be 3:2, 4:2, 5:5, 6:2, or 7:2.

Other proportions of mRNA encoding HIV Env protein and mRNA encoding lentivirus Gag protein are contemplated herein. In some embodiments, the immunogenic compositions comprise a lipid nanoparticle comprising mRNA encoding a membrane-bound HIV Env protein and a mRNA encoding a lentivirus Gag protein, wherein the ratio of the mRNA encoding a lentivirus Gag protein to the mRNA encoding a membrane-bound HIV Env protein is at least 1:1. For example, the ratio of the mRNA encoding a lentivirus Gag protein to the mRNA encoding a membrane-bound HIV Env protein may be 1:1, 2:1, 3:1, 4:1, or 5:1. In some embodiments, the ratio of the ratio of the mRNA encoding a lentivirus Gag protein to the mRNA encoding a membrane-bound HIV Env protein is at least 3:2. For example, the ratio of the mRNA encoding a lentivirus Gag protein to the mRNA encoding a membrane-bound HIV Env protein may be 3:2, 4:2, 5:5, 6:2, or 7:2.

In some embodiments, a single dose of an HIV mRNA vaccine of the present disclosure comprises 100 μg to 1000 μg of mRNA. For example, a single dose (e.g., comprising mRNA encoding HIV Env and mRNA encoding Gag formulated in a lipid nanoparticle at a ratio of Env:Gag of 3:2) may be 100 μg to 900 μg, 100 μg to 800 μg, 100 μg to 700 μg, 100 μg to 600 μg, 100 μg to 500 μg, 200 μg to 900 μg, 200 μg to 800 μg, 200 μg to 700 μg, 200 μg to 600 μg, 200 μg to 500 μg, 300 μg to 900 μg, 300 μg to 800 μg, 300 μg to 700 μg, 300 μg to 600 μg, or 300 μg to 600 μg. In some embodiments, a single dose (e.g., comprising mRNA encoding HIV Env and mRNA encoding Gag formulated in a lipid nanoparticle at a ratio of mRNA Env:mRNA Gag of 3:2) is 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 300 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, or 400 μg.

The HIV mRNA vaccines of the present disclosure are administered as multiple doses according to particular dosing schedule described herein. In some embodiments, a single initial dose is administered, followed by multiple booster doses. The amount of mRNA in an initial dose, in some embodiments, is less than the amount of mRNA in a subsequent booster dose.

A single dose (e.g., an initial dose) of an HIV mRNA vaccine, as provided herein, in some embodiments comprises 300 μg to 500 μg, or 350 to 450 μg, of mRNA encoding an HIV Env protein of one Clade (e.g., HIV Clade B Env protein, e.g., strain WITO, X2278, JRCSF, JR-FL, B41, 3988, 45_01dG5, BX08, RHPA, TRJO, YU2, or REJO) and mRNA encoding lentivirus Gag protein (e.g., formulated in a lipid nanoparticle at a ratio of mRNA Env:mRNA Gag of 3:2). In some embodiments, a single dose of an HIV mRNA vaccine comprises 400 μg of mRNA encoding an HIV Env protein of one Clade (e.g., HIV Clade B Env protein, e.g., strain WITO, X2278, JRCSF, JR-FL, B41, 3988, 45_01dG5, BX08, RHPA, TRJO, YU2, or REJO) and mRNA encoding lentivirus Gag protein (e.g., formulated in a lipid nanoparticle at a ratio of mRNA Env:mRNA Gag of 3:2). It should be understood that the initial dose may include an HIV Clade A Env protein. HIV Clade AC Env protein. HIV Clade AE Env protein, HIV Clade AG Env protein, an HIV Clade B Env protein, an HIV Clade C Env protein, HIV Clade D Env protein, HIV Clade G Env protein, or any other HIV Group M Env protein.

In some embodiments, a single dose (e.g., a booster dose) of an HIV mRNA vaccine comprises 150 μg to 350 μg, or 200 μg to 300 μg, of mRNA encoding an HIV Env protein of a first Clade (e.g., HIV Clade B Env protein, e.g., strain WITO) and mRNA encoding lentivirus Gag protein (e.g., formulated in a lipid nanoparticle at a ratio of mRNA Env:mRNA Gag of 3:2). In some embodiments, a single dose of an HIV mRNA vaccine comprises 240 μg, of mRNA encoding an HIV Env protein of a first Clade (e.g., HIV Clade B Env protein, e.g., strain WITO) and mRNA encoding lentivirus Gag protein (e.g., formulated in a lipid nanoparticle at a ratio of mRNA Env:mRNA Gag of 3:2). It should be understood that any booster dose may include an HIV Clade A Env protein, an HIV Clade AC Env protein, an HIV Clade AE Env protein, an HIV Clade AG Env protein, an HIV Clade B Env protein, an HIV Clade C Env protein, an HIV Clade D Env protein, an HIV Clade G Env protein, or any other HIV Group M Env protein. In some embodiments, a booster dose includes an Env protein of an HIV Clade that is the same as the initial dose. In some embodiments, a booster dose includes an Env protein of an HIV Clade that is different from the initial dose.

In some embodiments, a single dose (e.g., a booster dose) of an HIV mRNA vaccine comprises 150 μg to 350 μg, or 200 μg to 300 μg, of mRNA encoding an HIV Env protein of another Clade (e.g., HIV Clade A Env protein, e.g., strain BG505, Q23, Q842, MI369, KER2008, 0330, RW020, or B1369) and mRNA encoding lentivirus Gag protein (e.g., formulated in a lipid nanoparticle at a ratio of mRNA Env:mRNA Gag of 3:2). In some embodiments, a single dose of an HIV mRNA vaccine comprises 240 μg, of mRNA encoding an HIV Env protein of another Clade (e.g., HIV Clade A Env protein, e.g., strain BG505, Q23, Q842, MI369, KER2008, 0330, RW020, or BI369) and mRNA encoding lentivirus Gag protein (e.g., formulated in a lipid nanoparticle at a ratio of mRNA Env:mRNA Gag of 3:2).

In some embodiments, a single dose (e.g., a booster dose) of an HIV mRNA vaccine comprises 150 μg to 350 μg, or 200 μg to 300 μg, of mRNA encoding an HIV Env protein of yet another Clade (e.g., HIV Clade C Env protein, e.g., strain DU422, 426C, CH505, ZM176, ZM249, ZA012, DU156, CH848, CH1012, MM24, MM45, 001428, BR025, or MW965) and mRNA encoding lentivirus Gag protein (e.g., formulated in a lipid nanoparticle at a ratio of mRNA Env:mRNA Gag of 3:2). In some embodiments, a single dose of an HIV mRNA vaccine comprises 240 μg, of mRNA encoding an HIV Env protein of yet another Clade (e.g., HIV Clade C Env protein, e.g., strain DU422, 426C, CH505, ZM176, ZM249, ZA012, DU156, CH848, CH1012, MM24, MM45, 001428, BR025, or MW965) and mRNA encoding lentivirus Gag protein (e.g., formulated in a lipid nanoparticle at a ratio of mRNA Env:mRNA Gag of 3:2).

In some embodiments, two booster doses are administered simultaneously, e.g., intramuscularly, one in each arm. For example, following an initial 350 μg to 450 μg dose of a vaccine comprising mRNA encoding HIV Clade B Env protein and mRNA encoding lentivirus Gag protein, two booster doses of vaccine may be administered at the same time (e.g., on the same day, e.g., within hours or minutes of each other). One of the two booster doses may include, for example, mRNA encoding HIV Clade A Env protein and mRNA encoding lentivirus Gag protein, and the other of the two booster doses may include, for example, mRNA encoding HIV Clade C Env protein and mRNA encoding lentivirus Gag protein.

As discussed in the Examples, the present disclosure contemplates, in some embodiments, administration of a final low-dose booster. Thus, in some embodiments, a single booster dose of an HIV mRNA vaccine comprises 20 μg to 50 μg of mRNA encoding HIV Env protein and mRNA encoding lentivirus Gag protein. In some embodiments, a single booster dose of an HIV mRNA vaccine comprises 20 μg, 25 μg, 30 μg, 35 μg, 40 μg, 45 μg, or 50 μg of mRNA encoding HIV Env protein and mRNA encoding lentivirus Gag protein.

In some embodiments, the HIV vaccine therapies provided herein also include one or more boost dose of an HIV protein formulation. For example, an HIV protein boost dose may include 25 μg to 500 μg, or 50 μg to 200 μg (e.g., 50 μg, 100 μg, or 200 μg), of soluble HIV Env protein (e.g., SOSIP Env) and/or lentivirus Gag protein, without or without adjuvant (e.g., Adjuplex, or other adjuvant). In some embodiments, a protein boost dose is administered at least one month following an initial mRNA dose. For example, a protein boost dose may be administered at Week 45, 46, 47, 48, 49, 50, 51, 52, 53, 54, 55, 56, 57, 58, 59, or 60 of a vaccine dosing schedule.

### Lipid Nanoparticle (LNPs)

HIV RNA (e.g., mRNA) vaccines of the disclosure are formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

Vaccines of the present disclosure are typically formulated in lipid nanoparticle. In some embodiments, the lipid nanoparticle comprises at least one ionizable cationic lipid, at least one non-cationic lipid, at least one sterol, and/or at least one polyethylene glycol (PEG)-modified lipid.

In some embodiments, the lipid nanoparticle comprises a molar ratio of 20-60% ionizable cationic lipid. For example, the lipid nanoparticle may comprise a molar ratio of 20-50%, 20-40%, 20-30%, 30-60%, 30-50%, 30-40%, 40-60%, 40-50%, or 50-60% ionizable cationic lipid. In some embodiments, the lipid nanoparticle comprises a molar ratio of 20%, 30%, 40%, 50, or 60% ionizable cationic lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or 25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid.

In some embodiments, an ionizable cationic lipid of the disclosure comprises a compound having structure:

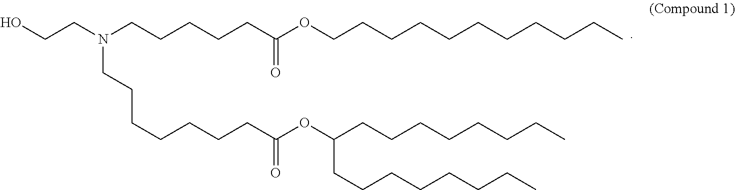 [SMILES: CCCCCCCCCCCOC(=O)CCCCCN(CCO)CCCCCCCC(=O)OC(CCCCCCCC)CCCCCCCC]

In some embodiments, an ionizable cationic lipid of the disclosure comprises a compound having structure:

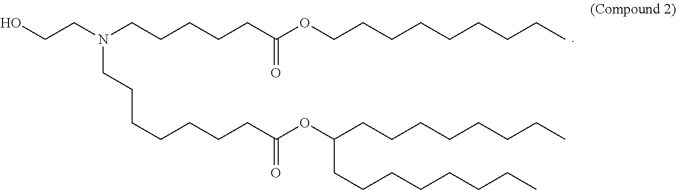 [SMILES: CCCCCCCCCOC(=O)CCCCCN(CCO)CCCCCCCC(=O)OC(CCCCCCCC)CCCCCCCC]

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, brassicasterol, tomatidine, ursolic acid, alpha-tocopherol, and mixtures thereof.

In some embodiments, a LNP of the disclosure comprises an ionizable cationic lipid of Compound 1, wherein the non-cationic lipid is DSPC, the structural lipid that is cholesterol, and the PEG lipid is PEG-DMG.

In some embodiments, a LNP of the disclosure comprises an N:P ratio of from about 2:1 to about 30:1.

In some embodiments, a LNP of the disclosure comprises an N:P ratio of about 6:1.

In some embodiments, a LNP of the disclosure comprises an N:P ratio of about 3:1.

In some embodiments, a LNP of the disclosure comprises a wt/wt ratio of the ionizable cationic lipid component to the RNA of from about 10:1 to about 100:1.

In some embodiments, a LNP of the disclosure comprises a wt/wt ratio of the ionizable cationic lipid component to the RNA of about 20:1.

In some embodiments, a LNP of the disclosure comprises a wt/wt ratio of the ionizable cationic lipid component to the RNA of about 10:1.

In some embodiments, a LNP of the disclosure has a mean diameter from about 50 nm to about 150 nm.

In some embodiments, a LNP of the disclosure has a mean diameter from about 70 nm to about 120 nm.

### Dosing Schedules/Immunization Protocols

The HIV mRNA vaccination methods of the present disclosure, in some embodiments, include administration of an initial dose of the vaccine followed by multiple (e.g., heterologous) booster doses, typically separated by at least one week. Herein, an initial dose formulated with an HIV Env of a particular Clade (e.g., Clade B) may be referred to as an “autologous dose,” whereas a subsequent booster dose formulated with an HIV Env of a different Clade (e.g., Clade A or C) may be referred to as a “heterologous dose.”

In some embodiments, an autologous dose of an HIV mRNA vaccine formulation is administered one or more time(s) within a particular time interval (the first time interval), and then multiple heterologous doses of an HIV mRNA vaccine formulation is administered one or more time(s) within a subsequent time interval (the second time interval). For example, an autologous dose of an HIV mRNA vaccine comprising mRNA encoding HIV Clade B Env (e.g., WITO) and mRNA encoding lentivirus Gag may be administered every 8-12 (e.g., 8, 9, 10, 11, or 12) weeks for 5-7 (e.g., 5, 6, or 7) months, and a heterologous dose of an HIV mRNA vaccine comprising mRNA encoding HIV Clade A Env (e.g., BG505, Q23, Q842, MI369, KER2008, 0330, RW020, or BI369) and mRNA encoding lentivirus Gag, and/or a heterologous dose of an HIV mRNA vaccine comprising mRNA encoding HIV Clade C Env (e.g., DU422, 426C, CH505, ZM176, ZM249, ZA012, DU156, CH848, CH1012, MM24, MM45, 001428, BR025, or MW965) and mRNA encoding lentivirus Gag, may be administered every 8-12 weeks for an additional 5-7 months. Thus, the present disclosure encompasses sequential immunizations initially with mRNA from a first Clade (e.g., Clade B) transmitter/founder envelope (e.g., WITO, X2278, JRCSF, JR-FL, B41, 3988, 45_01dG5, BX08, RHPA, TRJO, YU2, or REJO) followed by mixed heterologous envelopes from 2 different Clades (e.g., Clade A and Clade C), each co-formulated with mRNA encoding lentivirus Gag.

In some embodiments, the interval of time separating an initial dose from a booster dose, and/or separating one booster dose from another booster dose, is 2 to 10 weeks, or 2 to 15 weeks. For example, the interval of time separating an initial dose from a booster dose, and/or separating one booster dose from another booster dose, may be 2, 3, 4, 5, 6, 7, 8, 9, 10, 11, 12, 13, 14, or 15 weeks.

In some embodiments, a first lipid nanoparticle vaccine formulation is administered as multiple doses separated by at least 1 week per administration, prior to administration of a second lipid nanoparticle vaccine formulation. In some embodiments, a second lipid nanoparticle vaccine formulation is administered as multiple doses separated by at least 1 week per administration, after administration of a first lipid nanoparticle vaccine formulation. In some embodiments, a second lipid nanoparticle vaccine formulation and the at least one additional lipid nanoparticle vaccine formulation are administered simultaneously.

In some embodiments, an initial dose is administered (Week 0), and one or more booster dose is administered 10 to 60 weeks later. For example, as shown in the Examples, an initial dose may be administered at Week 0, and then subsequent heterologous booster doses administered at Weeks 11, 19, 27, 35, 43, 47, 51, and/or 56. In some embodiments, an initial dose may be administered at Week 0, and then subsequent heterologous booster doses administered at any one of Weeks 10-12 (e.g., 10, 11, or 12), 18-20 (e.g., 18, 19, or 20), 26-28 (e.g., 26, 27, or 28), 34-36 (e.g., 34, 35, or 36), 42-44 (e.g., 42, 43, or 44), 46-48 (e.g., 46, 47, or 48), 50-52 (e.g., 50, 51, or 52), and/or 55-57 (e.g., 55, 56, or 57). In some embodiments, an initial dose may be administered at Week 0, and then subsequent heterologous booster doses administered at any one of Weeks 5-10, 15-20, 25-30, 35-40, 45-50, and/or 55-60.

In some embodiments, a heterologous booster dose of an HIV mRNA vaccine is administered every 5 weeks, every 6 weeks, every 7 weeks, every 8 weeks, every 9 weeks, or every 10 weeks, for example, for at least 20, at least 22, at least 24, at least 26, at least 28, at least 30, at least 32, at least 34, at least 36, at least 38, at least 40, at least 42, at least 44, at least 46, at least 48, or at least 50 weeks. For example, a heterologous booster dose of an HIV mRNA vaccine may be administered every 5 weeks for at least 20, at least 22, at least 24, at least 26, at least 28, at least 30, at least 32, at least 34, at least 36, at least 38, at least 40, at least 42, at least 44, at least 46, at least 48, or at least 50 weeks. In some embodiments, a heterologous booster dose of an HIV mRNA vaccine may be administered every 6 weeks for at least 20, at least 22, at least 24, at least 26, at least 28, at least 30, at least 32, at least 34, at least 36, at least 38, at least 40, at least 42, at least 44, at least 46, at least 48, or at least 50 weeks. In some embodiments, a heterologous booster dose of an HIV mRNA vaccine may be administered every 7 weeks for at least 20, at least 22, at least 24, at least 26, at least 28, at least 30, at least 32, at least 34, at least 36, at least 38, at least 40, at least 42, at least 44, at least 46, at least 48, or at least 50 weeks. In some embodiments, a heterologous booster dose of an HIV mRNA vaccine may be administered every 8 weeks for at least 20, at least 22, at least 24, at least 26, at least 28, at least 30, at least 32, at least 34, at least 36, at least 38, at least 40, at least 42, at least 44, at least 46, at least 48, or at least 50 weeks. In some embodiments, a heterologous booster dose of an HIV mRNA vaccine may be administered every 9 weeks for at least 20, at least 22, at least 24, at least 26, at least 28, at least 30, at least 32, at least 34, at least 36, at least 38, at least 40, at least 42, at least 44, at least 46, at least 48, or at least 50 weeks. In some embodiments, a heterologous booster dose of an HIV mRNA vaccine may be administered every 10 weeks for at least 20, at least 22, at least 24, at least 26, at least 28, at least 30, at least 32, at least 34, at least 36, at least 38, at least 40, at least 42, at least 44, at least 46, at least 48, or at least 50 weeks.

Alternating intervals of time may also be used, for example, a booster at 10 weeks, a booster at 5 weeks, a booster at 10 weeks, a booster at 5 weeks, and so on.

In some embodiments, a 300-500 μg dose of an HIV mRNA vaccine is administered at Week 0, a 200-300 μg heterologous booster dose of an HIV mRNA vaccine is administered at any one of Weeks 10-12, a 200-300 μg heterologous booster dose of an HIV mRNA vaccine is administered at Weeks 18-20, a 200-250 μg heterologous booster dose of HIV mRNA vaccine is administered at Weeks 28-28, a 200-250 μg heterologous booster dose of an HIV mRNA vaccine is administered at Weeks 34-36, a 200-250 μg heterologous booster dose of an HIV mRNA vaccine is administered at Weeks 42-44, a 200-250 μg heterologous booster dose of an HIV mRNA vaccine is administered at Weeks 46-48, and a 30-50 μg heterologous booster dose of an HIV mRNA vaccine is administered at Weeks 55-57. In some embodiments, a 200-300 μg dose of an HIV protein vaccine is administered at Weeks 50-52.

In some embodiments, a 400 μg dose of HIV mRNA vaccine is administered at Week 0, a 240 μg heterologous booster dose of HIV mRNA vaccine is administered at Week 11, a 240 μg heterologous booster dose of HIV mRNA vaccine is administered at Week 19, a 225 sg heterologous booster dose of HIV mRNA vaccine is administered at Week 27, a 225 μg heterologous booster dose of HIV mRNA vaccine is administered at Week 35, a 225 μg heterologous booster dose of HIV mRNA vaccine is administered at Week 43, a 225 μg heterologous booster dose of HIV mRNA vaccine is administered at Week 47, and a 35 μg heterologous booster dose of HIV mRNA vaccine is administered at Week 56. In some embodiments, an HIV a 100 μg dose of protein vaccine is administered at Week 51.

In some embodiments, an initial dose of an HIV mRNA vaccine comprising mRNA encoding HIV Env (from a first Clade) and mRNA encoding lentivirus Gag is administered at Week 0, a booster dose of an HIV mRNA vaccine comprising mRNA encoding HIV Env (from the first Clade) and mRNA encoding lentivirus Gag is administered at any one of Weeks 10-12, a booster dose of an HIV mRNA vaccine comprising mRNA encoding HIV Env (from the first Clade) and mRNA encoding lentivirus Gag is administered at any one of Weeks 18-20, at least one heterologous booster dose of an HIV mRNA vaccine comprising mRNA encoding HIV Env (from a second Clade, and optionally from a third Clade) and mRNA encoding lentivirus Gag is administered at any one of Weeks 26-28, a heterologous booster dose of an HIV mRNA vaccine comprising mRNA encoding HIV Env (from the second Clade, and optionally from the third Clade) and mRNA encoding lentivirus Gag is administered at any one of Weeks 34-36, at least one heterologous booster dose of an HIV mRNA vaccine comprising mRNA encoding HIV Env (from the second Clade, and optionally from the third Clade) and mRNA encoding lentivirus Gag is administered at any one of Weeks 42-44, at least one heterologous booster dose of an HIV mRNA vaccine comprising mRNA encoding HIV Env (from the second Clade, and optionally from the third Clade) and mRNA encoding lentivirus Gag is administered at any one of Weeks 46-48, and optionally at least one heterologous booster dose of an HIV mRNA vaccine comprising mRNA encoding HIV Env (from the second Clade, and optionally from the third Clade) and mRNA encoding lentivirus Gag is administered at any one of Weeks 55-57.

In some embodiments, the methods comprise administering to the subject a first lipid nanoparticle comprising a mRNA encoding a first HIV Env protein and a mRNA encoding an HIV Gag polyprotein, administering to the subject a second lipid nanoparticle comprising a mRNA encoding a second HIV Env protein and a mRNA encoding an HIV Gag polyprotein, and administering to the subject a third lipid nanoparticle comprising a mRNA encoding a third HIV Env protein and a mRNA encoding an HIV Gag polyprotein, wherein the population of neutralizing antibodies comprises neutralizing antibodies that bind to shared epitopes on proteins from multiple different HIV strains. In some embodiments, at least one of the first, second, and third HIV Env proteins comprises a sequence of an Env protein of an HIV strain obtained from an infected subject who has broad and potent neutralizing antibodies to HIV Env protein. In some embodiments, at least one of the first, second, and third HIV Env proteins comprises a consensus sequence of variants an Env protein of an HIV strain obtained from an infected subject who has broad and potent neutralizing antibodies to HIV Env protein.

In some embodiments, the methods comprise administering to the subject a first lipid nanoparticle comprising a mRNA encoding an HIV Clade B Env protein and a mRNA encoding an HIV Gag polyprotein, administering to the subject a second lipid nanoparticle comprising a mRNA encoding an HIV Clade A Env protein and a mRNA encoding an HIV Gag polyprotein, and administering to the subject a third lipid nanoparticle comprising a mRNA encoding an HIV Clade C Env protein and a mRNA encoding an HIV Gag polyprotein, wherein the population of neutralizing antibodies comprises neutralizing antibodies that bind to shared epitopes on HIV Clade B proteins, HIV Clade A proteins, and HIV Clade C proteins.

In some embodiments, the population comprises neutralizing antibodies that bind to shared epitopes on proteins from multiple Clade B HIV strains, neutralizing antibodies that bind to shared epitopes on proteins from multiple Clade A HIV strains, and neutralizing antibodies that bind to shared epitopes on proteins from multiple Clade C HIV strains. In some embodiments, the population comprises neutralizing antibodies that bind to shared epitopes on proteins from at least five (5) different HIV strains. For example, the population may comprise neutralizing antibodies that bind to shared epitopes on proteins from at least 10 different HIV strains. In some embodiments, the population comprises neutralizing antibodies that bind to shared epitopes on proteins from any of the following HIV strains: JRFL, WITO.33, BG505, AD8, 398F1, CNE8, CNE55, 25710, CE1176, X1632, TRO11, X2278, BJOXO2000, X2632, 246F3, CHI 19, CE0217, A3, 02, and A3/02.

In some embodiments, none of the first, second, or at least one additional lipid nanoparticles comprise mRNA encoding a soluble HIV Env protein.

### Broadly Neutralizing Antibodies and Vaccine Efficacy

Antibody-mediated neutralization of viruses is the direct inhibition of viral infectivity resulting from antibody docking to virus particles (Burton D R et al. Curr Top Microbiol Immunol 2001; 260: 109-143). The elicitation of a neutralizing-antibody response is a correlate of protection for many vaccines and contributes to long-lived protection against many viral infections (Plotkin S A et al. Clin Vaccine Immunol 2010; 17: 1055-1065). A potent antiviral response may select for variants that allow escape from antibody neutralization and/or effector functions. Neutralization escape mechanisms are diverse and include the selection of amino acid variation in antibody epitopes directly as well as the modulation of structural features to prevent antibody binding.

As is known in the art, the number of antibodies required to neutralize a virus, such as HIV-1, can vary. While in many cases, the neutralization threshold for structurally distinct groups of viruses correlates positively with virion size, in agreement with the “coating theory” (Burton D R et al. 2001), factors that determine the number of antibodies required for neutralization may vary among viruses with different structures, compositions, and entry mechanisms. For example, the small number of functional trimers on the HIV-1 surface allows neutralization with a stoichiometry much lower than that predicted for a virion of this size (Yang X et al. J Virol 2005; 79: 3500-3508; and Klasse P J et al. Virology 2007; 369: 245-262). See also VanBlargan L A et al. Microbiology and Molecular Biology Reviews 2016; 80(4): 989-1010, incorporated herein by reference.

A broad and potent neutralizing antibody response to HIV (e.g., HIV-1) is one that can inhibit infectivity of multiple strains of HIV (e.g., heterologous tier-2 isolates). In some embodiments, a broad and potent neutralizing antibody response can inhibit infectivity of at least 2, at least 3, at least 4, at least 5 strains, at least 10 strains, or at least 15 strains of HIV (e.g., HIV-1). In some embodiments, a broad and potent neutralizing antibody response can inhibit infectivity of 2, 3, 4, 5, 6, 7, 8, 9, 10, 11, 12, 13, 14, or 15, or more of the following HIV strains: JRFL, WITO.33. BG505, AD8, 398F1, CNE8, CNE55, 25710. CE1176, X1632, TRO11, X2278, BJOXO2000, X2632, 246F3, CH119, CE0217, A3, 02, and A3/02. In some embodiments, neutralizing antibodies titers are calculated as the inhibitor concentrations (IC₅₀) or reciprocal plasma/serum dilutions (ID50) causing a 50% reduction of relative light units. In some embodiments, a broad and neutralizing antibody response is characterized as having an ID₅₀ titer of greater than 20. For example, the ID50 titer may be greater than 25, 30, 35, 40, 45, 50, 55, 60, 65, 70, 75, 80, 85, 90, 95, or 100. In some embodiments, the ID₅₀ titer is greater than 50. In some embodiments, the ID₅₀ titer is greater than 100. “Shared epitopes” of HIV antigens, such as HIV Env protein, comprise a common sequence motif to which broadly neutralizing antibodies can bind. Herein, in some embodiments, as discussed throughout the disclosure, the HIV vaccination methods elicit broadly neutralizing antibodies that bind to shared epitopes across multiple strains (e.g., at least 2, at least 3, at least 5, at least 10, or at least 15 strains) of HIV (e.g., HIV-1). In some embodiments, a first lipid nanoparticle vaccine formulation and a second lipid nanoparticle vaccine formulation are administered more than once (e.g., 2 to 10 times, e.g., 2, 3, 4, 5, 6, 7, 8, 9, or 10 times, or more) and in an amount effective at inducing in the subject a population of neutralizing antibodies that bind to shared epitopes on proteins from a first HIV Clade and neutralizing antibodies that bind to shared epitopes on proteins from a (at least a) second HIV Clade. In some embodiments, the population comprises neutralizing antibodies that bind to shared epitopes on proteins from multiple strains (e.g., at least 2, at least 3, at least 5, at least 10, or at least 15 strains) of the first Clade and neutralizing antibodies that bind to shared epitopes on proteins from multiple strains (e.g., at least 2, at least 3, at least 5, at least 10, or at least 15 strains) of the second Clade.

In some embodiments, the methods further comprise administering to the subject at least one additional lipid nanoparticle comprising a mRNA encoding an HIV Env protein from at least one additional Clade (e.g., any one of Group M Clades A-K) and a mRNA encoding an HIV Gag protein. Thus, in some embodiments, the population comprises neutralizing antibodies that bind to shared epitopes on proteins from multiple strains of the at least one additional Clade (e.g., any one of Group M Clades A-K).

Some aspects of the present disclosure provide formulations of the HIV mRNA vaccine, wherein the HIV mRNA vaccine is formulated in an effective amount to produce an antigen specific immune response in a subject (e.g., production of antibodies specific to an HIV antigen). “An effective amount” is a dose of an HIV mRNA vaccine effective to produce an antigen-specific immune response. Also provided herein are methods of inducing an antigen-specific immune response in a subject.

In some embodiments, the antigen-specific immune response is characterized by measuring an anti-HIV antigen (e.g., anti-HIV Env and/or anti-HIV Gag) antibody titer produced in a subject administered an HIV mRNA vaccine as provided herein. An antibody titer is a measurement of the amount of antibodies within a subject, for example, antibodies that are specific to a particular antigen or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by the HIV mRNA vaccine.

The HIV therapies (e.g., combination of vaccine formulations and dosing schedule) provided herein produce in a subject broadly neutralizing antibodies against multiple HIV strains. In some embodiments, a broadly neutralizing antibody titer produced in a subject is increased by at least 1 log relative to a control. For example, a broadly neutralizing antibody titer produced in a subject may be increased by at least 1.5, at least 2, at least 2.5, or at least 3 log relative to a control. In some embodiments, a broadly neutralizing antibody titer produced in the subject is increased by 1, 1.5, 2, 2.5 or 3 log relative to a control. In some embodiments, a broadly neutralizing antibody titer produced in the subject is increased by 1-3 log relative to a control. For example, the a broadly neutralizing antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, a broadly neutralizing antibody titer produced in a subject is increased at least 2 times relative to a control. For example, a broadly neutralizing antibody titer produced in a subject may be increased at least 3 times, at least 4 times, at least 5 times, at least 6 times, at least 7 times, at least 8 times, at least 9 times, or at least 10 times relative to a control. In some embodiments, a broadly neutralizing antibody titer produced in the subject is increased 2, 3, 4, 5, 6, 7, 8, 9, or 10 times relative to a control. In some embodiments, a broadly neutralizing antibody titer produced in a subject is increased 2-10 times relative to a control. For example, a broadly neutralizing antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

A control may be, for example, an unvaccinated subject, or a subject administered a live attenuated HIV vaccine, an inactivated HIV vaccine, or a protein subunit HIV vaccine. A control, in some embodiments, is a broadly neutralizing antibody titer produced in a subject who has not been administered an HIV mRNA vaccine. In some embodiments, a control is a broadly neutralizing antibody titer produced in a subject administered a recombinant or purified HIV protein vaccine. Recombinant protein vaccines typically include protein antigens that either have been produced in a heterologous expression system (e.g., bacteria or yeast) or purified from large amounts of the pathogenic organism.

In some embodiments, an effective amount of an HIV mRNA vaccine is a dose that is reduced compared to the standard of care dose of a recombinant HIV protein vaccine. A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a recombinant or purified HIV protein vaccine, or a live attenuated or inactivated HIV vaccine, or an HIV VLP vaccine, that a physician/clinician or other medical professional would administer to a subject to treat or prevent HIV, or an HIV-related condition, while following the standard of care guideline for treating or preventing HIV, or an HIV-related condition.

In some embodiments, a broadly neutralizing antibody titer produced in a subject administered an effective amount of an HIV mRNA vaccine is equivalent to a broadly neutralizing antibody titer produced in a control subject administered a standard of care dose of a recombinant or purified HIV protein vaccine, or a live attenuated or inactivated HIV vaccine, or an HIV VLP vaccine.

In some embodiments, an effective amount of an HIV mRNA vaccine is a dose equivalent to an at least 2-fold reduction in a standard of care dose of a recombinant or purified HIV protein vaccine. For example, an effective amount of an HIV mRNA vaccine may be a dose equivalent to an at least 3-fold, at least 4-fold, at least 5-fold, at least 6-fold, at least 7-fold, at least 8-fold, at least 9-fold, or at least 10-fold reduction in a standard of care dose of a recombinant or purified HIV protein vaccine. In some embodiments, an effective amount of an HIV mRNA vaccine is a dose equivalent to an at least at least 100-fold, at least 500-fold, or at least 1000-fold reduction in a standard of care dose of a recombinant or purified HIV protein vaccine. In some embodiments, an effective amount of an HIV mRNA vaccine is a dose equivalent to a 2-, 3-, 4-, 5-, 6-, 7-, 8-, 9-, 10-, 20-, 50-, 100-, 250-, 500-, or 1000-fold reduction in a standard of care dose of a recombinant or purified HIV protein vaccine. In some embodiments, a broadly neutralizing antibody titer produced in a subject administered an effective amount of an HIV mRNA vaccine is equivalent to a broadly neutralizing antibody titer produced in a control subject administered the standard of care dose of a recombinant or protein HIV protein vaccine, or a live attenuated or inactivated HIV vaccine, or an HIV VLP vaccine. In some embodiments, an effective amount of an HIV mRNA vaccine is a dose equivalent to a 2-fold to 1000-fold (e.g., 2-fold to 100-fold, 10-fold to 1000-fold) reduction in the standard of care dose of a recombinant or purified HIV protein vaccine, wherein a broadly neutralizing antibody titer produced in the subject is equivalent to a broadly neutralizing antibody titer produced in a control subject administered the standard of care dose of a recombinant or purified HIV protein vaccine, or a live attenuated or inactivated HIV vaccine, or an HIV VLP vaccine.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

Efficacy=(ARU−ARV)/ARU×100; and

Efficacy=(1−RR)×100.

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

Effectiveness=(1−OR)×100.

In some embodiments, efficacy of the HIV vaccine is at least 60% relative to unvaccinated control subjects. For example, efficacy of the HIV vaccine may be at least 65%, at least 70%, at least 75%, at least 80%, at least 85%, at least 95%, at least 98%, or 100% relative to unvaccinated control subjects.

Sterilizing Immunity. Sterilizing immunity refers to a unique immune status that prevents effective viral infection into the host. In some embodiments, the effective amount of an HIV vaccine of the present disclosure is sufficient to provide sterilizing immunity in the subject for at least 1 year. For example, the effective amount of an HIV vaccine of the present disclosure is sufficient to provide sterilizing immunity in the subject for at least 2 years, at least 3 years, at least 4 years, or at least 5 years. In some embodiments, the effective amount of an HIV vaccine of the present disclosure is sufficient to provide sterilizing immunity in the subject at an at least 5-fold lower dose relative to control. For example, the effective amount may be sufficient to provide sterilizing immunity in the subject at an at least 10-fold lower, 15-fold, or 20-fold lower dose relative to a control.

Detectable Antigen. In some embodiments, the effective amount of an HIV vaccine of the present disclosure is sufficient to produce detectable levels of HIV antigen as measured in serum of the subject at 1-72 hours post administration.

Titer. An antibody titer is a measurement of the amount of antibodies within a subject, for example, antibodies that are specific to a particular antigen (e.g., an anti-HIV antigen). Antibody titer is typically expressed as the inverse of the greatest dilution that provides a positive result. Enzyme-linked immunosorbent assay (ELISA) is a common assay for determining antibody titers, for example.

In some embodiments, the effective amount of an HIV vaccine of the present disclosure is sufficient to produce a 1,000-10,000 neutralizing antibody titer produced by neutralizing antibody against the HIV antigen as measured in serum of the subject at 1-72 hours post administration. In some embodiments, the effective amount is sufficient to produce a 1,000-5,000 neutralizing antibody titer produced by neutralizing antibody against the HIV antigen as measured in serum of the subject at 1-72 hours post administration. In some embodiments, the effective amount is sufficient to produce a 5,000-10,000 neutralizing antibody titer produced by neutralizing antibody against the HIV antigen as measured in serum of the subject at 1-72 hours post administration.

In some embodiments, the neutralizing antibody titer is at least 100 neutralizing units per milliliter (NU/mL). For example, the neutralizing antibody titer may be at least 200, 300, 400, 500, 600, 700, 800, 900 or 1000 NU/mL. In some embodiments, the neutralizing antibody titer is at least 10,000 NU/mL.

In some embodiments, a geometric mean, which is the nth root of the product of n numbers, is generally used to describe proportional growth. Geometric mean, in some embodiments, is used to characterize antibody titer produced in a subject.

mRNA Design, Elements, and Manufacturing

Messenger RNA (mRNA) is any ribonucleic acid that encodes a (at least one) protein (a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents RNA (e.g., mRNA), the “T”s would be substituted for “U”s. Thus, any of the DNAs disclosed and identified by a particular sequence identification number herein also disclose the corresponding RNA (e.g., mRNA) sequence complementary to the DNA, where each “T” of the DNA sequence is substituted with “U.”

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs, but that those elements, unlike the ORF, need not necessarily be present in a vaccine of the present disclosure. It is contemplated that the HIV vaccines of the present disclosure comprise at least one (one or more) ribonucleic acid (RNA) having an open reading frame encoding at least one HIV antigen.

In some embodiments, the RNA is a messenger RNA (mRNA) having an open reading frame encoding at least one HIV antigen. In some embodiments, the RNA (e.g., mRNA) further comprises a (at least one) 5′ UTR, 3′ UTR, a polyA tail and/or a 5′ cap.

mRNA of the present disclosure, in some embodiments, is not naturally-occurring. That is, the mRNA in some embodiments, is engineered, for example, chemically synthesized or produced using recombinant nucleic acid technology. In some embodiments, at least one mRNA of the compositions provided herein is naturally-occurring.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a vaccine of the disclosure. Non-limiting examples of 5′ UTR sequences include

(SEQ ID NO: 10)
GGGAAAUAAGAGAGAAAAGAAGAGUAAGAAGAAAUAUAAGAGCCACC
and
(SEQ ID NO: 11)
GGGAAAUAAGAGAGAAAAGAAGAGUAAGAAGAAAUAUAAGACCCCGG
CGCCGCCACC

A “3′ untranslated region” (UTR) refers to a region of an mRNA that is directly downstream (i.e., 3′) from the stop codon (i.e., the codon of an mRNA transcript that signals a termination of translation) that does not encode a polypeptide. 3′ UTR: Non-limiting examples of 3′ UTR sequences include

(SEQ ID NO: 12)
UGAUAAUAGGCUGGAGCCUCGGUGGCCAUGCUUCUUGCCCCUUGGGCC
UCCCCCCAGCCCCUCCUCCCCUUCCUGCACCCGUACCCCCGUGGUCUU
UGAAUAAAGUCUGAGUGGGCGGC
and
(SEQ ID NO: 13)
UGAUAAUAGGCUGGAGCCUCGGUGGCCUAGCUUCUUGCCCCUUGGGCC
UCCCCCCAGCCCCUCCUCCCCUUCCUGCACCCGUACCCCCGUGGUCUU
UGAAUAAAGUCUGAGUGGGCGGC

An “open reading frame” is a continuous stretch of DNA beginning with a start codon (e.g., methionine (ATG)), and ending with a stop codon (e.g., TAA, TAG or TGA) and encodes a polypeptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, the length of a mRNA is 200 to 3,000 nucleotides. For example, the length of a mRNA may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

### Sequence Optimization

In some embodiments, an ORF encoding an Env and/or Gag protein of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding: bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% (e.g., less than 90%, less than 85%, less than 80%, or less than 75%) sequence identity to a naturally-occurring or wild-type sequence ORF (e.g., a naturally-occurring or wild-type mRNA sequence encoding an ENV or Gag protein). In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding an ENV or Gag protein).

In some embodiments, a codon optimized mRNA may be one in which the levels of G/C are enhanced. The G/C-content of nucleic acid molecules (e.g., mRNA) may influence the stability of the mRNA. mRNA having an increased amount of guanine (G) and/or cytosine (C) residues may be functionally more stable than mRNA containing a large amount of adenine (A) and thymine (T) or uracil (U) nucleotides. As an example, WO2002098443 (published Dec. 12, 2002) discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemically Unmodified Nucleotides

In some embodiments, at least one RNA (e.g., mRNA) of HIV vaccines of the present disclosure is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Chemical Modifications

HIV mRNA vaccines of the present disclosure comprise, in some embodiments, at least one mRNA having an open reading frame encoding at least one Env and/or Gag protein, wherein the mRNA comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB2017/051367 all of which are incorporated by reference herein.

Thus, mRNA of the disclosure can comprise standard nucleotides and nucleosides, naturally-occurring nucleotides and nucleosides, non-naturally-occurring nucleotides and nucleosides, or any combination thereof.

The mRNA in some embodiments, comprises non-natural modified nucleotides that are introduced during synthesis or post-synthesis of the mRNA to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a mRNA may be chemically modified.

The present disclosure provides for modified nucleosides and nucleotides of a mRNA. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. mRNA can comprise a region or regions of linked nucleosides. Such regions may have variable backbone linkages. The linkages can be standard phosphodiester linkages, in which case the mRNA would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in the mRNA having at least one chemical modification. One example of such non-standard base pairing is the base pairing between the modified nucleotide inosine and adenine, cytosine or uracil. Any combination of base/sugar or linker may be incorporated into mRNA of the present disclosure.

In some embodiments, modified nucleobases in mRNA comprise I-methyl-pseudouridine (m1ψ), 1-ethyl-pseudouridine (e1ψ), 5-methoxy-uridine (mo5U), 5-methyl-cytidine (m5C), and/or pseudouridine (W). In some embodiments, modified nucleobases in mRNA comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a MRNA of the disclosure comprises 1-methyl-pseudouridine (m1ψ) substitutions at one or more or all uridine positions of the mRNA. In some embodiments, a MRNA of the disclosure comprises 1-methyl-pseudouridine (m1ψ) substitutions at one or more or all uridine positions of the mRNA and 5-methyl cytidine substitutions at one or more or all cytidine positions of the mRNA. In some embodiments, a MRNA of the disclosure comprises pseudouridine (ψ) substitutions at one or more or all uridine positions of the mRNA. In some embodiments, a MRNA of the disclosure comprises pseudouridine (ψ) substitutions at one or more or all uridine positions of the mRNA and 5-methyl cytidine substitutions at one or more or all cytidine positions of the mRNA. In some embodiments, a mRNA of the disclosure comprises uridine at one or more or all uridine positions of the mRNA.

In some embodiments, mRNA is uniformly modified (e.g., fully modified, modified throughout the entire sequence) for a particular modification. For example, mRNA can be uniformly modified with 1-methyl-pseudouridine, meaning that all uridine residues in the mRNA sequence are replaced with 1-methyl-pseudouridine. Similarly, mRNA can be uniformly modified for any type of nucleoside residue present in the sequence by replacement with a modified residue such as those set forth above.

The mRNA may contain from about 1% to about 100% modified nucleotides (either in relation to overall nucleotide content, or in relation to one or more types of nucleotide, i.e., any one or more of A, G, U or C) or any intervening percentage. It will be understood that any remaining percentage is accounted for by the presence of unmodified A, G, U, or C. The mRNA may contain at a minimum 1% and at maximum 100% modified nucleotides, or any intervening percentage. In some embodiments, at least 5%, at least 10%, at least 25%, at least 50%, at least 80%, at least 90% or 100% of the uracil in the mRNA is replaced with a modified uracil (e.g., a 5-substituted uracil).

### In Vitro Transcription of RNA

cDNA encoding the polynucleotides described herein may be transcribed using an in vitro transcription (IVT) system. In vitro transcription of RNA is known in the art and is described in WO2014/152027, which is incorporated by reference herein in its entirety.

In some embodiments, the mRNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of HIV mRNA. In some embodiments, cells. e.g., bacterial cells. e.g., E. coli, e.g., DH-1 cells are transfected with the plasmid DNA template. In some embodiments, the transfected cells are cultured to replicate the plasmid DNA which is then isolated and purified. In some embodiments, the DNA template includes a RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the RNA comprises 5′ terminal cap, for example, 7mG(5′)ppp(5′)NlmpNp.

### Chemical Synthesis

Solid-phase chemical synthesis. Nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis. The synthesis of nucleic acids of the present disclosure by the sequential addition of monomer building blocks may be carried out in a liquid phase.

Combination of Synthetic Methods. The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNATM oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the nucleic acids of the present invention may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Additional Embodiments

1. A method of inducing in a subject an immune response to human immunodeficiency virus (HIV), the method comprising:
(a) during a first period of time, administering to a subject an initial dose and multiple autologous boost doses of a first composition comprising a messenger RNA (mRNA) encoding an HIV envelope (Env) protein and an mRNA encoding a lentivirus group-specific antigen (Gag) protein formulated in a lipid nanoparticle;
(b) during a second period of time, administering to the subject multiple heterologous boost doses of a second composition comprising an mRNA encoding an HIV Env protein and an mRNA encoding a lentivirus Gag protein formulated in a lipid nanoparticle; and
(c) producing in the subject a broadly neutralizing antibody response against multiple strains of HIV.

2. The method of paragraph 1, wherein the broadly neutralizing antibody response comprises a production of neutralizing antibodies that bind to shared epitopes on proteins from the multiple strains of HIV.

3. The method of paragraph 2, wherein the broadly neutralizing antibody response comprises an ID₅₀ titer of greater than 20 or greater than 50.

4. The method of paragraph 1, wherein (i) the first period of time is 1-30 weeks following administration of the initial dose of the first composition and/or (ii) the second period of time is 8-60 weeks following administration of the initial dose of the first composition.

5. The method of paragraph 1, wherein the time between any two doses of the first composition of (a) and/or the second composition of (b) is at least 1 week, is at least 4 weeks, or is 4-12 weeks.

6. The method of paragraph 1, wherein the HIV is HIV type-1 (HIV-1).

7. The method of paragraph 1, wherein the ratio of the mRNA encoding an HIV Env protein to the mRNA encoding an HIV Gag protein of in the first composition of (a) and/or in the second composition of (b) is 2:1 or 3:2.

8. The method of paragraph 1, wherein the first composition and/or the second composition further comprises an mRNA encoding an HIV protease.

9. The method of paragraph 8, wherein the ratio of the mRNA encoding an HIV Gag protein to the mRNA encoding an HIV protease to is at least 1:5, at least 1:10, at least 1:20, at least 1:40, at least 1:60, or at least 1:80.

10. The method of paragraph 1, wherein the first and/or second composition further comprises an mRNA encoding furin.

11. The method of paragraph 10, wherein the ratio of the mRNA encoding furin to the mRNA encoding an HIV Env protein is at least 1:5.

12. The method of paragraph 1, wherein the HIV Env protein of the first composition of (a) is selected from an HIV Env protein of Group M Clade A-K, wherein the HIV Env protein of the second composition of (b) is selected from an HIV Env protein of Group M Clade A-K, and wherein the Clade(s) of the HIV Env protein of the first composition of (a) is different from the Clade(s) of the HIV Env protein of the second composition of (b).

13. The method of paragraph 1, wherein the HIV Env protein of the first composition of (a) and/or the second composition of (b) comprises HIV Env SOSIP.664 mutations.

14. The method of paragraph 1, wherein the HIV Env protein of the first composition of (a) and/or the second composition of (b) is a membrane-bound HIV Env protein.

15. The method of paragraph 1, wherein the cytosolic portion of the HIV Env protein of the first composition of (a) and/or the second composition of (b) is partially truncated.

16. The method of paragraph 14, wherein the membrane-bound HIV Env protein is gp150 or gp160.

17. The method of paragraph 1, wherein the HIV Env protein of the first composition of (a) and/or the second composition of (b) comprises a sequence of an Env protein, or a consensus sequence of variants an Env protein, of an HIV strain obtained from an infected subject who has broadly neutralizing antibodies to HIV Env protein.

18. The method of paragraph 1, wherein the HIV Env protein of the first composition of (a) and/or the second composition of (b) comprises a sequence of a variant Env protein of an HIV strain that engages one or multiple UCA antibodies to HIV Env protein from the lineages of known broadly neutralizing antibodies.

19. The method of paragraph 1, wherein the HIV Env protein of the first composition of (a) and/or the second composition of (b) comprises:
(i) a mutation selected from 153E, 190G and N276D, relative to strain WITO4160.27 HIV Env protein, or other mutations suitable to remove the glycans at positions 188 and 276, optionally wherein the HIV Env protein of the first composition of (a) and/or the second composition of (b) further comprises a disulfide bridge at 113C-432GCG;
(ii) a mutation selected from N460D and N463D relative to strain BG505 HIV Env protein, or other suitable mutations to remove the glycans at positions 460 and 463;
(iii) a mutation selected from K295N, D386N, and 375Y, relative to strain DU422.1, C DU156.12, DU172.17, ZM176.66, or CNE58 HIV Env protein, optionally wherein the HIV Env protein of the first composition of (a) and/or the second composition of (b) further comprises a disulfide bridge at 133C-432GCG; or
(iv) a mutation selected from T322N and S375Y, relative to strain WITO4160.27 HIV Env protein, optionally wherein the HIV Env protein of the first composition of (a) and/or the second composition of (b) further comprises a disulfide bridge at 113C-429GCG.

20. The method of paragraph 1, wherein the HIV Env protein of the first composition of (a) and/or the second composition of (b) is a tier-2 Env with all the major glycan holes filled in by insertion of the missing glycans.

21. The method of paragraph 1, wherein the lentivirus is selected from human immunodeficiency virus (HIV), simian immunodeficiency virus (SIV), and murine leukemia virus (muLV).

22. The method of paragraph 1, wherein the lentivirus-derived Gag is replaced by VSV-AG or VSV core protein.

23. A method of inducing in a subject an immune response to human immunodeficiency virus (HIV), the method comprising:
administering to the subject a first lipid nanoparticle comprising a messenger RNA (mRNA) encoding an HIV envelope (Env) protein from a first Clade and an mRNA encoding a lentivirus group-specific antigen (Gag) protein; and
administering to the subject a second lipid nanoparticle comprising a mRNA encoding an HIV Env protein from a second Clade and a mRNA encoding a lentivirus Gag protein,
wherein the first lipid nanoparticle and the second lipid nanoparticle are administered more than once and in an amount effective at inducing in the subject a population of neutralizing antibodies that bind to shared epitopes on proteins from the first Clade and neutralizing antibodies that bind to shared epitopes on proteins from the second Clade.

24. The method of paragraph 23, wherein the HIV is HIV type-1.

25. The method of paragraph 23, wherein the population comprises neutralizing antibodies that bind to shared epitopes on proteins from multiple strains of the first Clade and neutralizing antibodies that bind to shared epitopes on proteins from multiple strains of the second Clade.

26. The method of paragraph 23 further comprising administering to the subject at least one additional lipid nanoparticle comprising an mRNA encoding an HIV Env protein from at least one additional Clade and a mRNA encoding an HIV Gag protein.

27. The method of paragraph 26, wherein the population comprises neutralizing antibodies that bind to shared epitopes on proteins from multiple strains of the at least one additional Clade.

28. The method of paragraph 23, wherein the first nanoparticle comprises a ratio of the mRNA encoding an HIV Env protein to the mRNA encoding an HIV Gag protein of at least 1:1, and/or wherein the second nanoparticle comprises a ratio the mRNA encoding an HIV Env protein to the mRNA encoding an HIV Gag protein of at least 1:1.

29. The method of paragraph 28, wherein the first nanoparticle comprises a ratio of the mRNA encoding an HIV Env protein to the mRNA encoding an HIV Gag protein of at least 3:2, and/or wherein the second nanoparticle comprises a ratio the mRNA encoding an HIV Env protein to the mRNA encoding an HIV Gag protein of at least 3:2.

30. The method of paragraph 23, wherein the HIV Env protein comprises mutations, relative to wild-type HIV Env protein, that favor a closed conformation.

31. The method of paragraph 23, wherein the HIV Env protein comprises glycan knock-in or knock-out modifications.

32. The method of paragraph 23, wherein the HIV Env protein is a stabilized soluble Env protein.

33. The method of paragraph 32, wherein the HIV Env protein is an HIV Env SOSIP.664 protein.

34. The method of paragraph 23, wherein the HIV Env protein is a membrane-bound HIV Env protein.

35. The method of paragraph 34, wherein the cytosolic portion of the protein is truncated.

36. The method of paragraph 34, wherein the membrane-bound HIV Env protein is gp150 or gp160.

37. The method of paragraph 23, wherein the lentivirus is selected from human immunodeficiency virus (HIV), simian immunodeficiency virus (SIV), and murine leukemia virus (muLV).

38. The method of paragraph 23, wherein the first Clade, the second Clade, and the at least one additional Clade are selected from the group consisting of HIV Group M Clades A-K and related circulating recombinant forms (CRFs).

39. The method of paragraph 23, wherein the first lipid nanoparticle and the second nanoparticle are administered sequentially.

40. The method of paragraph 39, wherein the first lipid nanoparticle is administered as multiple doses separated by at least 1 week per administration, prior to administration of the second lipid nanoparticle.

41. The method of paragraph 39, wherein the second lipid nanoparticle is administered as multiple doses separated by at least 1 week per administration, after administration of the first lipid nanoparticle.

42. The method of paragraph 23, wherein the second lipid nanoparticle and the at least one additional lipid nanoparticle are administered simultaneously.

43. The method of paragraph 38, wherein the first Clade is Clade A.

44. The method of paragraph 38, wherein the second Clade is Clade A.

45. The method of paragraph 43 or 44, wherein the HIV Env protein is selected from HIV Clade A BG505, Q23, Q842, MI369, KER2008, 0330, RW020, and B1369 strain Env proteins.

46. The method of paragraph 38, wherein the first Clade is Clade AC.

47. The method of paragraph 38, wherein the second Clade is Clade AC.

48. The method of paragraph 46 or 47, wherein the HIV Env protein is an HIV Clade AC 3301 strain Env proteins.

49. The method of paragraph 38, wherein the first Clade is Clade AE.

50. The method of paragraph 38, wherein the second Clade is Clade AE.

51. The method of paragraph 49 or 50, wherein the HIV Env protein is selected from HIV Clade AE C2101, CM244, and BJOXO28000 strain Env proteins.

52. The method of paragraph 38, wherein the first Clade is Clade AG.

53. The method of paragraph 38, wherein the second Clade is Clade AG.

54. The method of paragraph 52 or 53, wherein the HIV Env protein is selected from HIV Clade AG DJ263 and T280 strain Env proteins.

55. The method of paragraph 38, wherein the first Clade is Clade B.

56. The method of paragraph 38, wherein the second Clade is Clade B.

57. The method of paragraph 55 or 56, wherein the HIV Env protein is selected from HIV Clade B WITO, X2278, JRCSF, JR-FL, B41, 3988, 45_01dG5, BX08, RHPA, TRJO, YU2, and REJO strain Env proteins.

58. The method of paragraph 38, wherein the first Clade is Clade C.

59. The method of paragraph 38, wherein the second Clade is Clade C.

60. The method of paragraph 58 or 59, wherein the HIV Env protein is selected from HIV Clade C DU422, 426C, CH505, ZM176, ZM249, ZA012, DU156, CH848, CH1012, MM24, MM45, 001428, BR025, and MW965 strain Env proteins.

61. The method of paragraph 38, wherein the first Clade is Clade D.

62. The method of paragraph 38, wherein the second Clade is Clade D.

63. The method of paragraph 61 or 62, wherein the HIV Env protein is an HIV Clade D A07412M1 strain Env protein.

64. The method of paragraph 38, wherein the first Clade is Clade G.

65. The method of paragraph 38, wherein the second Clade is Clade G.

66. The method of paragraph 64 or 65, wherein the HIV Env protein is selected from HIV Clade G X1193 and P1981 strain Env proteins.

67. The method of paragraph 23, wherein the HIV Env protein comprises a sequence of an Env protein of an HIV strain obtained from an infected subject who has broadly neutralizing antibodies to HIV Env protein.

68. The method of paragraph 23, wherein the HIV Env protein comprises a consensus sequence of variants an Env protein of an HIV strain obtained from an infected subject who has broadly neutralizing antibodies to HIV Env protein.

69. The method of paragraph 23, wherein the method comprises
administering to the subject a first lipid nanoparticle comprising a mRNA encoding a first HIV Env protein and a mRNA encoding an HIV Gag polyprotein;
administering to the subject a second lipid nanoparticle comprising a mRNA encoding a second HIV Env protein and a mRNA encoding an HIV Gag polyprotein; and
administering to the subject a third lipid nanoparticle comprising a mRNA encoding a third HIV Env protein and a mRNA encoding an HIV Gag polyprotein;
wherein the population of neutralizing antibodies comprises neutralizing antibodies that bind to shared epitopes on proteins from multiple different HIV strains.

70. The method of paragraph 69, wherein at least one of the first, second, and third HIV Env proteins comprises a sequence of an Env protein of an HIV strain obtained from an infected subject who has broadly neutralizing antibodies to HIV Env protein.

71. The method of paragraph 69, wherein at least one of the first, second, and third HIV Env proteins comprises a consensus sequence of variants an Env protein of an HIV strain obtained from an infected subject who has broadly neutralizing antibodies to HIV Env protein.

72. The method of paragraph 23, wherein the method comprises
administering to the subject a first lipid nanoparticle comprising a mRNA encoding an HIV Clade B Env protein and a mRNA encoding an HIV Gag polyprotein;
administering to the subject a second lipid nanoparticle comprising a mRNA encoding an HIV Clade A Env protein and a mRNA encoding an HIV Gag polyprotein; and
administering to the subject a third lipid nanoparticle comprising a mRNA encoding an HIV Clade C Env protein and a mRNA encoding an HIV Gag polyprotein;
wherein the population of neutralizing antibodies comprises neutralizing antibodies that bind to shared epitopes on HIV Clade B proteins, HIV Clade A proteins, and HIV Clade C proteins.

73. The method of paragraph 72, wherein the population comprises neutralizing antibodies that bind to shared epitopes on proteins from multiple Clade B HIV strains, neutralizing antibodies that bind to shared epitopes on proteins from multiple Clade AN HIV strains, and neutralizing antibodies that bind to shared epitopes on proteins from multiple Clade C HIV strains.

74. The method of paragraph 23, wherein the population comprises neutralizing antibodies that bind to shared epitopes on proteins from at least 5 different HIV strains.

75. The method of paragraph 74, wherein the population comprises neutralizing antibodies that bind to shared epitopes on proteins from at least 10 different HIV strains.

76. The method of paragraph 75, wherein the population comprises neutralizing antibodies that bind to shared epitopes on proteins from any of the following HIV strains: JRFL, WITO.33, BG505, AD8, 398F1, CNE8, CNE55, 25710, CE1176, X1632, TRO11, X2278, BJOXO2000, X2632, 246F3, CH119, CE0217, A3, 02, and A3/02.

77. The method of paragraph 23, wherein none of the first, second, or at least one additional lipid nanoparticles comprise mRNA encoding a soluble HIV Env protein.

78. An immunogenic composition, comprising a lipid nanoparticle comprising mRNA encoding a membrane-bound HIV Env protein and a mRNA encoding an HIV Gag protein, wherein the lipid nanoparticle comprises an ionizable cationic lipid, a non-cationic lipid, sterol, and a PEG-modified lipid.

79. An immunogenic composition, comprising a lipid nanoparticle comprising mRNA encoding an HIV Env SOSIP.664 protein and a mRNA encoding an HIV Gag protein.

80. An immunogenic composition, comprising a lipid nanoparticle comprising mRNA encoding a membrane-bound HIV Env protein and a mRNA encoding an HIV Gag protein, wherein the ratio of the mRNA encoding a membrane-bound HIV Env protein to the mRNA encoding an HIV Gag protein is at least 3:2.

81. The method of any one of the preceding paragraphs, wherein the Env protein encoded by the mRNA of the first composition and the Env protein encoded by the mRNA of the second composition are from difference clades.

82. The method of any one of the preceding paragraphs, wherein the first composition the second composition are administered more than once and in an amount effective at inducing in the subject a population of neutralizing antibodies that bind to shared epitopes on HIV Env proteins from different Clades.

83. The method of any one of the preceding paragraphs, wherein the first composition comprises mRNA encoding an HIV Env protein selected from HIV Clade A Env proteins, HIV Clade AC Env proteins, HIV Clade AE Env proteins, HIV Clade AG Env proteins, HIV Clade B Env proteins, HIV Clade C Env proteins, HIV Clade D Env proteins, and HIV Clade G Env proteins.

84. The method of any one of the preceding paragraphs, wherein the first composition comprises a first mRNA and a second mRNA, each encoding an HIV Env protein selected from HIV Clade A Env proteins, HIV Clade AC Env proteins, HIV Clade AE Env proteins, HIV Clade AG Env proteins, HIV Clade B Env proteins, HIV Clade C Env proteins, HIV Clade D Env proteins, and HIV Clade G Env proteins, wherein the Env protein encoded by the first mRNA and the Env protein encoded by the second mRNA are from different clades.

85. The method of any one of the preceding paragraphs, wherein the first composition comprises a first mRNA, a second mRNA, and a third mRNA, each encoding an HIV Env protein selected from HIV Clade A Env proteins, HIV Clade AC Env proteins, HIV Clade AE Env proteins, HIV Clade AG Env proteins, HIV Clade B Env proteins. HIV Clade C Env proteins, HIV Clade D Env proteins, and HIV Clade G Env proteins, wherein the Env protein encoded by the first mRNA, the Env protein encoded by the second mRNA, and the Env protein encoded by the third mRNA are from different clades.

86. The method of any one of the preceding paragraphs, wherein the second composition comprises mRNA encoding an HIV Env protein selected from HIV Clade A Env proteins, HIV Clade AC Env proteins, HIV Clade AE Env proteins, HIV Clade AG Env proteins, HIV Clade B Env proteins, HIV Clade C Env proteins, HIV Clade D Env proteins, and HIV Clade G Env proteins, and wherein the Env proteins encoded by the mRNA of the first composition and the Env proteins encoded by the mRNA of the second composition are from different clades.

87. The method of any one of the preceding paragraphs, wherein the second composition comprises a first mRNA and a second mRNA, each encoding an HIV Env protein selected from HIV Clade A Env proteins, HIV Clade AC Env proteins, HIV Clade AE Env proteins, HIV Clade AG Env proteins, HIV Clade B Env proteins, HIV Clade C Env proteins, HIV Clade D Env proteins, and HIV Clade G Env proteins, wherein the Env protein encoded by the first mRNA and the Env protein encoded by the second mRNA are from different clades, and wherein the Env proteins encoded by the mRNA of the first composition and the Env proteins encoded by the mRNA of the second composition are from different clades.

88. The method of any one of the preceding paragraphs, wherein the second composition comprises a first mRNA, a second mRNA, and a third mRNA, each encoding an HIV Env protein selected from HIV Clade A Env proteins, HIV Clade AC Env proteins, HIV Clade AE Env proteins, HIV Clade AG Env proteins, HIV Clade B Env proteins, HIV Clade C Env proteins, HIV Clade D Env proteins, and HIV Clade G Env proteins, wherein the Env protein encoded by the first mRNA, the Env protein encoded by the second mRNA, and the Env protein encoded by the third mRNA are from different clades, and wherein the Env proteins encoded by the mRNA of the first composition and the Env proteins encoded by the mRNA of the second composition are from different clades.

89. The method of any one of the preceding paragraphs, wherein the HIV Clade A Env proteins are selected from HIV Clade A BG505, Q23, Q842. MI369. KER2008, 0330, RW020 or B1369 strain Env proteins.

90. The method of any one of the preceding paragraphs, wherein the HIV Clade AC Env proteins are HIV Clade AC 3301 strain Env proteins.

91. The method of any one of the preceding paragraphs, wherein the HIV Clade AE Env proteins are selected from HIV Clade AE C2101, CM244, and BJOXO28000 strain Env proteins.

92. The method of any one of the preceding paragraphs, wherein the HIV Clade AG Env proteins are selected from HIV Clade AG DJ263 and T280 strain Env proteins.

93. The method of any one of the preceding paragraphs, wherein the HIV Clade B Env proteins are selected from HIV Clade B X2278. JRCSF, JR-FL, B41, 3988, 45_01dG5, BX08, RHPA, TRJO, YU2, and REJO strain Env proteins.

94. The method of any one of the preceding paragraphs, wherein the HIV Clade C Env proteins are selected from HIV Clade C DU422, 426C, CH505, ZM176, ZM249, ZA012, DU156, CH848, CH1012, MM24, MM45, 001428, BR025, and MW965 strain Env proteins.

95. The method of any one of the preceding paragraphs, wherein the HIV Clade D Env proteins are selected from HIV Clade D A07412M1 strain Env proteins.

96. The method of any one of the preceding paragraphs, wherein the HIV Clade G Env proteins are selected from HIV Clade G X1193 and P1981 strain Env proteins.

97. The method of any one of the preceding paragraphs, wherein the RNA comprises a chemical modification.

98. The method of any one of the preceding paragraphs, wherein the chemical modification is 1-methylpseudouridine.

99. The method of any one of the preceding paragraphs, wherein the lipid nanoparticle comprises a PEG-modified lipid, a non-cationic lipid, a sterol, an ionizable cationic lipid, or any combination thereof.

100. The method of any one of the preceding paragraphs, wherein the lipid nanoparticle comprises 0.5-15% PEG-modified lipid; 5-25% non-cationic lipid; 25-55% sterol; and 20-60% ionizable cationic lipid.

101. The method of any one of the preceding paragraphs, wherein the PEG-modified lipid is 1,2 dimyristoyl-sn-glycerol, methoxypolyethyleneglycol (PEG2000 DMG), the non-cationic lipid is 1,2 distearoyl-sn-glycero-3-phosphocholine (DSPC), the sterol is cholesterol; and the ionizable cationic lipid has the structure of Compound 1.

### EXAMPLES

### Example 1: Multi-Clade mRNA Immunizations Spur Cross Clade Immune Response

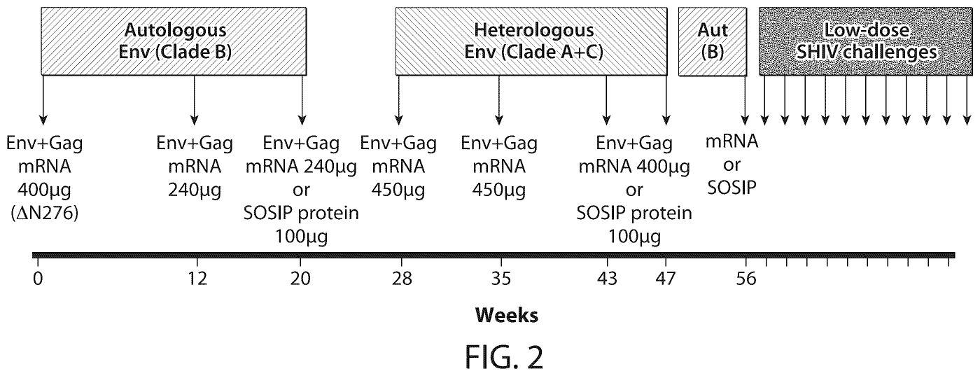

Fifteen Rhesus macaques were divided into Groups (“GR”). Two groups (GRs. 3 and 4) received seven doses of immunizations containing mRNA encoding an HIV-1 envelope protein, with either a Native or Modified CD4 receptor-binding ability, co-formulated with mRNA encoding an S1V group-specific antigen (Gag) protein (Table 1). The other two groups (GRs. 1 and 2) received five doses of the same formula and two doses of pre-made soluble HIV-1 Env protein (SOSIP trimer) administered with an adjuvant (Adjuplex). The immunizations were delivered approximately every eight weeks (FIG. 2), intramuscularly carried by the lipid nanoparticle comprising an ionizable cationic lipid of Compound 1.

The first two doses contained mRNA encoding an Env protein from Clade B. These were followed by either a third dose of the same formula (GRs. 3 and 4) or a dose of the soluble envelope proteins (FIG. 2). Three more doses of mRNA encoding proteins from Clades A and C were then administered followed by a either a fourth dose of the same formula (GRs. 3 and 4) or a dose of soluble envelope proteins (FIG. 2). Two additional immunizations of Clade B followed. A single protein boost of soluble envelope protein from Clade B was administered at week 51 and a dose sparing boost of either mRNA encoding an Env or a soluble envelope protein was administered at week 56 (FIG. 2, Table1).

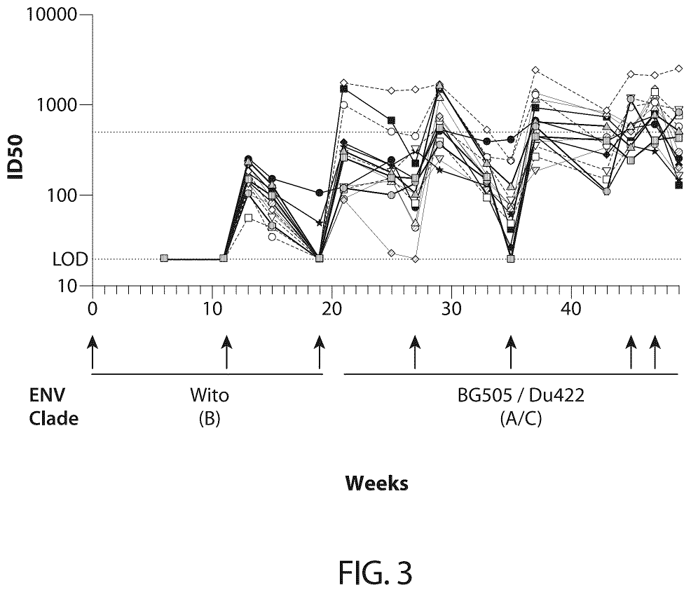

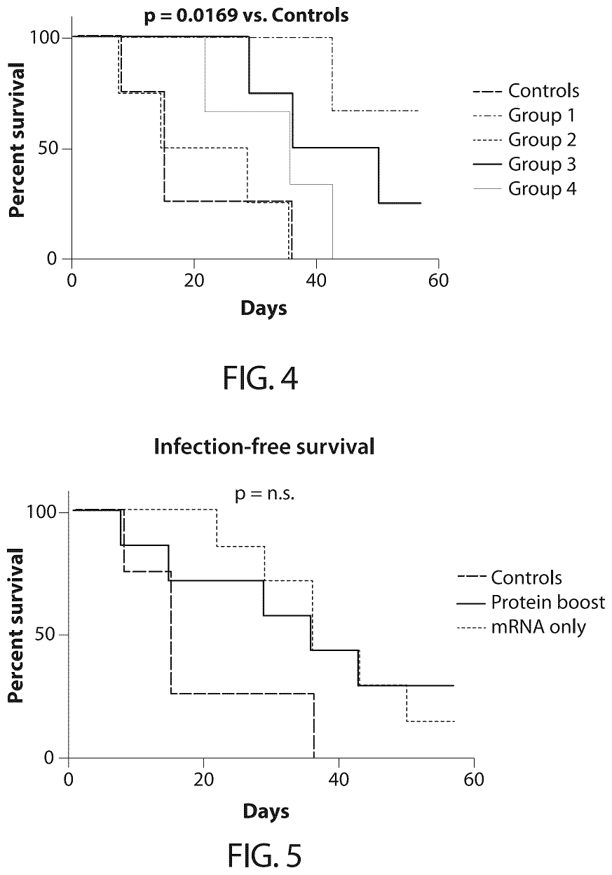

Blood draws were taken and neutralization titers were performed against autologous HIV-1 strains starting at week 13 (after the second dose) as well as a broad-spectrum neutralization of heterologous HIV-1 strains at week 58 (Tables 2 and 3). As Table 2 shows, there is an increasing immunological response to the strain from Clade B, which response persists and increases beyond the administration of the doses containing the strain. This can also be seen in FIG. 3, where the ID₅₀ increases over time with respect to autologous HIV-1. Moreover, as Table 3 shows, the immunizations confer an immunological response against not only the strains administered, but against a global panel of strains, including strains from Clades outside of the immunization group. Finally, vaccinated macaques were challenged with live tier-2 heterologous virus (SHIV ADS) by repeated low-dose inoculations at weekly intervals (Table 4). An additional group of four naïve macaques were also infected as controls. Immunized animals showed a partial resistance to infection as they remained uninfected or showed a delayed infection relative to the control group (FIG. 4). Protection for animals who received mRNA followed by protein boosts (GRs. 1 and 2) did not statistically differ from protection observed in animals who received only mRNA (Grs. 3 and 4, FIGS. 4 and 5; p=ns)

TABLE 1
Study groups with immunization compositions

| | | | | | | | | | | | JR-FL | WITO |
|---|---|---|---|---|---|---|---|---|---|---|---|---|
| | | | Env | WITO (B) | | | BG505 (A)/Du422 (C) | | | | (B) | (B) |
| | GR | n | Form | Wk 0 | Wk 11 | Wk 19 | Wk 27 | Wk 35 | Wk 43 | Wk 47 | Wk 51 | Wk 56 |
| mRNA | 1 | n = 3 | WT | mRNA | mRNA | Protein | mRNA | mRNA | mRNA | Protein | Protein | Protein |
| prime + | | | | 400 ug | 240 ug | 100 ug | 225 ug | 225 ug | 225 ug | 200 ug | 100 ug | 50 ug |
| protein | | | | WITO: | WITO: | Soluble | BG505: | BG505: | BG505: | Soluble | Soluble | Soluble |
| boost | | | | Gag | Gag | SOSIP + | Gag | Gag | Gag | SOSIP + | SOSIP + | SOSIP + |
| | | | | | | Adjv | 225 ug | 225 ug | 225 ug | Adjv | Adjv | Adjv |
| | | | | | | WITO | DU422: | DU422: | DU422: | DU422 | JRFL | WITO |
| | | | | | | | Gag | Gag | Gag | BG505 | | |
| | 2 | n = 4 | DS | 400 ug | 240 ug | | mRNA | mRNA | mRNA | | | |
| | | | Locked | mRNA | mRNA | | 225 ug | 225 ug | 225 ug | | | |
| | | | | WITO: | WITO: | | BG505: | BG505: | BG505: | | | |
| | | | | Gag | Gag | | Gag | Gag | Gag | | | |
| | | | | | | | 225 ug | 225 ug | 225 ug | | | |
| | | | | | | | DU422: | DU422: | DU422: | | | |
| | | | | | | | Gag | Gag | Gag | | | |
| mRNA | 3 | n = 4 | WT | 400 ug | 240 ug | 240 ug | mRNA | mRNA | mRNA | 225 ug | Protein | 35 ug |
| prime + | | | | mRNA | mRNA | mRNA | 225 ug | 225 ug | 225 ug | mRNA | 100 ug | mRNA |
| mRNA | | | | WITO: | WITO: | WITO: | BG505: | BG505: | BG505: | BG505: | Soluble | WITO: |
| boost | | | | Gag | Gag | Gag | Gag | Gag | Gag | Gag | SOSIP + | Gag |
| | | | | | | | 225 ug | 250 ug | 225 ug | 225 ug | Adjv | |
| | | | | | | | DU422: | DU422: | DU422: | mRNA | JRFL | |
| | | | | | | | Gag | Gag | Gag | DU422: | | |
| | 4 | n = 4 | DS | 400 ug | 240 ug | | mRNA | mRNA | mRNA | Gag | | |
| | | | locked | mRNA | mRNA | | 225 ug | 225 ug | 225 ug | | | |
| | | | | WITO: | WITO: | | BG505: | BG505: | BG505: | | | |
| | | | | Gag | Gag | | Gag | Gag | Gag | | | |
| | | | | | | | 225 ug | 225 ug | 225 ug | | | |
| | | | | | | | DU422: | DU422: | DU422: | | | |
| | | | | | | | Gag | Gag | Gag | | | |

TABLE 2
Neutralization Titers against the autologous HIV-1 Strain (WITO)

| | | Immunizations: | | | | | | | | | | | |
|---|---|---|---|---|---|---|---|---|---|---|---|---|---|
| | | | | Week 11 | | | Week 19 | | | Week 27 | | | Week 35 |
| | | Week 0 | | WITO | | | WITO | | | BG + DU | | | BG + DU |
| Group | Animal ID | WITO | wk. 6 | wk. 11 | wk. 13 | wk. 15 | wk. 19 | wk. 21 | wk. 25 | wk. 27 | wk. 29 | wk. 33 | wk. 35 |
| 1 | 1D2 | WT | <20 | <20 | 165 | 47 | <20 | 521 | 10 [symbol] | 137 | [symbol] 1 | 147 | <20 |
| | AZ71 | mRNA + | <20 | <20 | 151 | 10 [symbol] | <20 | 205 | 162 | 154 | [symbol] 58 | 160 | <20 |
| | BH80 | SOSIP | <20 | <20 | 245 | [symbol] 4 | <20 | 317 | 179 | 107 | 1605 | 234 | 1 [symbol] |
| 2 | 7G4 | [symbol] | <20 | <20 | 23 [symbol] | 57 | <20 | 204 | 180 | 327 | 601 | 226 | 87 |
| | [symbol] | mRNA + | <20 | <20 | 12 [symbol] | 70 | <20 | [symbol] | 14 [symbol] 2 | 149 [symbol] | 1705 | 537 | 239 |
| | AZ25 | SOSIP | <20 | <20 | 113 | 35 | <20 | 1017 | 50 [symbol] | 457 | 1572 | 265 | 245 |
| | 37218 | | <20 | <20 | 57 | 45 | <20 | 11 [symbol] | 156 | 62 | 398 | [symbol] 5 | 49 |
| 3 | AZ97 | WI | <20 | <20 | 105 | 78 | <20 | 26 [symbol] | 146 | [symbol] 0 | 1227 | 168 | 74 |
| | BO23 | mRNA | <20 | <20 | 2 [symbol] 4 | [symbol] 1 | <20 | 122 | 145 | [symbol] | 24 [symbol] | [symbol] 13 | 8 [symbol] |
| | 37462 | | <20 | <20 | 134 | 63 | <20 | 50 | 23 | <20 | 753 | 140 | <20 |
| | 38730 | | <20 | <20 | 104 | 79 | <20 | 52 | 1 [symbol] 0 | 44 | 70 [symbol] | 142 | 7 [symbol] |
| 4 | AV55 | [symbol] | <20 | <20 | 2 [symbol] 7 | [symbol] | 50 | 349 | [symbol] | 305 | [symbol] 92 | 12 [symbol] | 60 |
| | BA07 | mRNA | <20 | <20 | 172 | [symbol] 0 | <20 | 3523 | [symbol] | 2 [symbol] 5 | [symbol] | 235 | 42 |
| | BV94 | | <20 | <20 | 1 [symbol] 5 | [symbol] | <20 | 385 | 220 | [symbol] 52 | 667 | 105 | 27 |
| | 39267 | | <20 | <20 | 25 [symbol] | 1 [symbol] | 107 | 122 | [symbol] 4 [symbol] | 74 | [symbol] 19 | 357 | 4 [symbol] |

| | | Immunizations: | | | | | | | | |
|---|---|---|---|---|---|---|---|---|---|---|
| | | | Week 43 | | Week 47 | | Week 51 | | Week 56 | |
| | | | BG + DU | | BG + DU | | JK + FL | | WITO | |
| Group | Animal ID | wk. 37 | wk. 43 | wk. 45 | wk. 47 | wk. 49 | wk. 51 | wk. 53 | wk. 56 | wk. 58 |
| 1 | 1D2 | [symbol] 65 | 112 | 1142 | 371 | 837 | 730 | 623 | 214 | 489 |
| | AZ71 | 447 | 40 [symbol] | 245 | 409 | 4 [symbol] 8 | 369 | 433 | 178 | 298 |
| | BH80 | 684 | 504 | 347 | 80 [symbol] | [symbol] 2 [symbol] | 510 | [symbol] | 153 | 59 [symbol] |
| 2 | 7G4 | 340 | 104 | 1105 | 1081 | [symbol] 7 [symbol] | 14 [symbol] 1 | 6 [symbol] 4 | 307 | 400 |
| | [symbol] | 2439 | 265 | 2207 | [symbol] | 25 [symbol] | 20 [symbol] | 1 [symbol] 87 | 11 [symbol] 4 | [symbol] 97 |
| | AZ25 | 1302 | 357 | 9 [symbol] 5 | 107 [symbol] | 570 | 41 [symbol] | 417 | [symbol] 9 [symbol] | [symbol] 19 [symbol] |
| | 37218 | [symbol] 72 | 150 | 30 [symbol] | 13 [symbol] | 7 [symbol] | [symbol] 93 | 94 [symbol] | 277 | 2144 |
| 3 | AZ97 | [symbol] | 52 [symbol] | 398 | [symbol] | [symbol] 40 | 443 | 8 [symbol] | 207 | 121 [symbol] |
| | BO23 | 185 | 5 [symbol] | 247 | 37 [symbol] | 164 | 290 | 432 | 202 | 603 |
| | 37462 | [symbol] 0 | 774 | 504 | 1504 | 2 [symbol] 2 | 553 | 6 [symbol] 5 | 173 | [symbol] 3 [symbol] |
| | 38730 | 382 | 435 | 521 | 1327 | 303 | 593 | 793 | 254 | [symbol] |
| 4 | AV55 | 4 [symbol] 4 | 109 | [symbol] | [symbol] 05 | [symbol] 49 | 232 | 42 [symbol] | 137 | [symbol] 82 |
| | BA07 | 92 [symbol] | 749 | 59 [symbol] | [symbol] 3 [symbol] | [symbol] | 278 | 40 [symbol] | 244 | 364 |
| | BV94 | 462 | 282 | 59 [symbol] | 766 | 220 | 304 | 1737 | 504 | [symbol] 34 |
| | 39267 | 670 | 411 | 35 [symbol] | 61 [symbol] | 2 [symbol] 7 | 4 [symbol] 9 | 274 | [symbol] 72 | 482 |

[symbol]

indicates data missing or illegible when filed

TABLE 3
Broad-Spectrum Neutralization of Heterologous Tier-2 HIV-1 Strains

| | | | Strain: | | | | | | | |
|---|---|---|---|---|---|---|---|---|---|---|
| | | | JRFL | WITO.33 | BG505 | AD8 | 398F1 | 246F3 | CNE8 | CNE55 |
| | | | Clade: | | | | | | | |
| | | | B | B | A | B | A | AC | AE | AE |
| PL No. | Group | Animal ID | wk. 58 | wk. 58 | wk. 58 | wk. 58 | wk. 58 | wk. 58 | wk. 58 | wk. 58 |
| 1 | 1 | 1D2 | 44 | 9 | 18 | 8 | 89 | 44 | 35 | <10 |
| 6 | 1 | AZ71 | 40 | 11 | 9 | 33 | 63 | 20 | 24 | <10 |
| 9 | 1 | BH80 | 52 | 34 | 18 | 44 | 114 | 52 | 51 | <10 |
| 2 | 1 | 7G4 | 60 | 14 | 24 | 33 | 40 | 36 | 36 | <10 |
| 3 | 1 | 8L4 | 54 | 68 | 39 | 36 | 48 | 24 | 21 | <10 |
| 5 | 1 | AZ25 | 68 | 32 | 25 | 34 | 79 | 23 | 38 | <10 |
| 12 | 2 | 37218 | 64 | 20 | 20 | 86 | 74 | 60 | 41 | 17 |
| 7 | 1 | AZ97 | 48 | 28 | 35 | 84 | 126 | 56 | 44 | <10 |
| 10 | 1 | BO23 | 48 | 21 | 220 | 46 | 44 | 38 | 36 | <10 |
| 13 | 1 | 37462 | 58 | 8 | 8 | 47 | 77 | 37 | 44 | 13 |
| 14 | 3 | 38730 | 32 | 20 | 27 | 82 | 79 | 39 | 41 | <10 |
| 4 | 4 | AV55 | 80 | 36 | 16 | 84 | 61 | 28 | 42 | <10 |
| 8 | 4 | BA07 | 43 | 26 | 52 | 50 | 36 | 14 | 35 | <10 |
| 11 | 4 | BV94 | 40 | 24 | 25 | 40 | 59 | 47 | 59 | <10 |
| 15 | 4 | 39267 | 64 | 31 | 70 | 42 | 175 | 72 | 60 | 16 |

| | | | Strain: | | | | | | | |
|---|---|---|---|---|---|---|---|---|---|---|
| | | | TRO11 | X2278 | BIOXO | CH119 | 25710 | CE1178 | CE0217 | X1632 |
| | | | Clade: | | | | | | | |
| | | | B | B | BC | BC | C | C | C | G |
| PL No. | Group | Animal ID | wk. 58 | wk. 58 | wk. 58 | wk. 58 | wk. 58 | wk. 58 | wk. 58 | wk. 58 |
| 1 | 1 | 1D2 | 35 | 42 | <10 | 53 | 35 | 31 | 15 | 16 |
| 6 | 1 | AZ71 | 46 | 38 | 20 | 20 | 31 | 22 | 12 | 9 |
| 9 | 1 | BH80 | 75 | 183 | 37 | 37 | 35 | 27 | 20 | 40 |
| 2 | 1 | 7G4 | 78 | <10 | <10 | 32 | 35 | 26 | 12 | <10 |
| 3 | 1 | 8L4 | 54 | 88 | 20 | 29 | 47 | 26 | 37 | 40 |
| 5 | 1 | AZ25 | 64 | <10 | <10 | 33 | 33 | 20 | 14 | 8 |
| 12 | 2 | 37216 | 61 | 112 | 56 | 56 | 53 | 34 | 36 | 20 |
| 7 | 1 | AZ97 | 80 | 44 | 40 | 43 | 61 | 29 | 20 | 12 |
| 10 | 1 | BO23 | 32 | <10 | 37 | 30 | 32 | 21 | 18 | 14 |
| 13 | 1 | 37462 | 48 | 80 | 76 | 56 | 46 | 33 | 25 | 42 |
| 14 | 3 | 38730 | 47 | 40 | 18 | 53 | 60 | 25 | 20 | 9 |
| 4 | 4 | AV55 | 80 | 28 | 21 | 41 | 45 | 35 | 40 | 68 |
| 8 | 4 | BA07 | 50 | 194 | 20 | 26 | 26 | 13 | 23 | <10 |
| 11 | 4 | BV94 | 58 | 160 | 28 | 35 | 33 | 32 | 29 | 17 |
| 15 | 4 | 39267 | 55 | 75 | 24 | 58 | 63 | 50 | 54 | 18 |

### Materials and Methods

### Monkeys

Fifteen female Rhesus macaques (Macaca mulatta), aged 6 to 12, were maintained in accordance with the guidelines of the Committee on Care and Use of Laboratory Animals (which is incorporated herein by reference) and housed in a biosafety level 2 facility at BIOQUAL, Inc. (Rockville, MD). The animals were grouped by carefully balancing age, weight, and complete blood count (CBC)/chemistry parameters. Four additional naïve macaques were included in the study during the challenge phase. All animals were negative for the major histocompatibility complex (MHC) class I Mamu-A*01 allele.

### Proteins

Soluble stabilized envelope proteins (SOSIP trimers) were produced in 293 Freestyle cells by transient co-transfection of plasmids encoding Env DNA and the cellular protease Furin. Cell culture supernatants were harvested 5 or 6 days after transfection. After 0.22 um filtering, the supernatants were sequentially loaded on a Galanthus nivalis lectin column, followed by passage on a size-exclusion column and then on a mAb 447-52D negative selection column. Finally, the purified trimers were concentrated to 1-2 mg/ml in PBS and stored at −80° C. Site-directed mutagenesis was performed to introduce specific mutations. The N188 glycan was introduced in Clade B WITO.33 and WITO.33 113C-432GCG SOSIP. Other SOSIP proteins included BG505 (Clade A) containing the following mutations: T332N, 241N, 289N, 375Y or BG505 T332N, 241N, 289N, 375Y, D113C-R429C; JR-FL (Clade B) containing the following mutations: 375Y and JR-FL, D113C-R432GCG 375Y; and DU422 (Clade C) containing the following mutations: 295N, 386N, 375Y and Du422 295N, 386N, 375Y, 113C-432GCG.

mRNA Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNATM oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

mRNA Quantification

In some embodiments, the nucleic acids of the present disclosure may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Viruses

In vivo-titrated SHIV_{AD8-EO} virus stocks were provided by Dr. Malcolm A. Martin (LMM, NIAID). Virus stocks were prepared by transfecting 293T cells with SHIV AD8-CK15 molecular clones using Lipofectamine 2000. Culture supernatants were collected 48 hours later. The virus stock infectivity was measured by infecting Con A-stimulated rhesus PBMCs and aliquots were stored at −80° C. until use.

For in vitro neutralization assays, HIV-1 pseudoparticles expressing wide-type or mutated gp160 from BG505 and other isolates were produced in HEK 293T cells by co-transfecting Env-expressing plasmids with a backbone plasmid, pSG3^{Δenv}, expressing a full-length HIV-1 clone with a defective env gene using Mirus293 Transfection Reagent. Culture supernatants were collected 48 hours later and aliquots were stored at −80° C. until use: virus stock infectivity titers were measured by using serial dilutions in TZM-bl cells.

### Experimental Replication, Randomization, and Blinding

For vaccination, 15 macaques were allocated to experimental groups in order to balance the average age, weight and peripheral WBC and CD4⁺ T-cell counts. Each in vitro experiment was independently performed at least twice in duplicate wells to ensure reproducibility.

### Lipid Nanoparticle

An ionizable cationic lipid nanoparticle formulation, comprising a molar ratio of 20-60% ionizable cationic lipid (Compound 1), 5-25% non-cationic lipid, 25-55% sterol, and 0.5-15% PEG-modified lipid, was used for these experiments.

### Dosing

All macaque immunizations were performed at BIOQUAL, Inc facility. All 15 macaques were immunized with 500 μl of the dosing material via the intramuscular route into the right posterior thigh of each animal. Injections included 500 μl of co-formulated Env+Gag mRNAs, or SOSIP.664 trimers (wild-type or interdomain-stabilized) pre-mixed with 100 μL of Adjuplex adjuvant (Sigma). Immunizations were performed at weeks 0, 11, 19, 27, 35, 43, 47, 51, 56, and 59.

### Blood Draws

Animals were sedated and blood was drawn from a posterior leg vein generally at 2 weeks after each immunization, as well as every week during the virus challenge phase. Plasma and PBMC were collected and stored frozen. An aliquot of blood was regularly sent to an external laboratory for blood chemistry and CBC counts.

### Virus Challenge

The SHIV_{AD8-EO} is a CCR5-tropic tier 2 (neutralization-sensitivity phenotype) pathogenic strain that replicates to high levels in rhesus macaques. The virus stock was titrated in macaque PBMC and diluted in PBS to 10 TCID₅₀ at the time of challenge. All animals were inoculated intra-rectally with low-dose (10 tissue culture infectious doses per dose) SHIV_{AD8-EO} at weekly intervals until infection became established. A 3 ml speculum was used to gently open the rectum, and a 1-ml suspension of virus in a tuberculin syringe was slowly infused into the rectal cavity.

### Titers

Macaque plasma samples were collected from two weeks post-immunization and other interested time points (e.g., plasma samples were collected at weeks −2, 7, 19, 27, 31, 35, 43, 47, 53, and 60). The neutralization was performed by using single-cycle infection of TZM-bl cells by ENV pseudoviruses. Serial dilutions of plasma samples were incubated with pseudotyped viruses for 30 minutes in 96 well plates and then 100 μl TZM-Bl cells that contain 10,000 cells were added. Reporter gene activation signal was detected at 48 hours later after removing 150 μl media and adding the 40 μl Luciferase Assay Reagent per well. Relative Light Unit were recorded and half-maximal inhibitory concentrations (IC₅₀) were performed using Graphpad Prism 7.

### Example 2

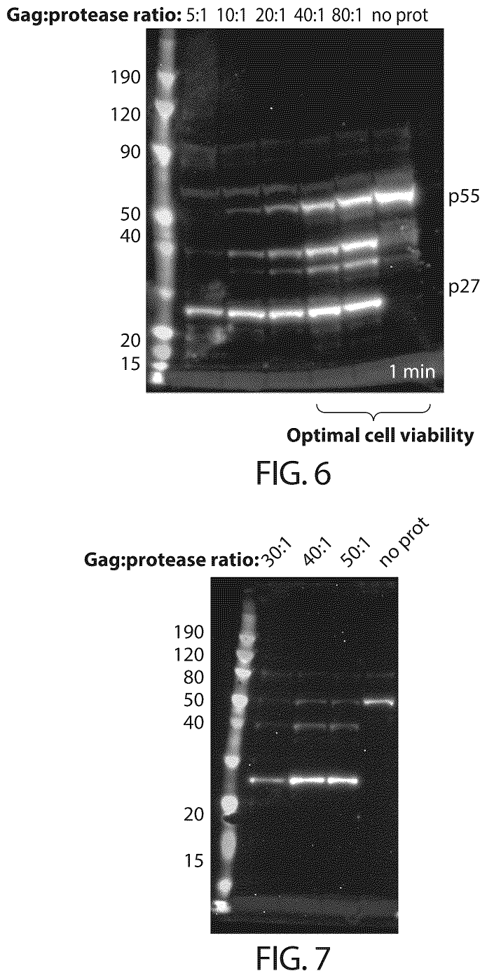

The data in this set of experiments shows that the SIV Gag polyprotein is efficiently processed to its final products, including the main core protein p27, in the presence of SIV protease (FIG. 6). The image in FIG. 6 shows a Western blot analysis of cell lysates obtained from 293-T human embryonic kidney cells stably transduced to express HIV-1 WITO.153E Env using a retroviral vector. The cells were transfected with SIV Gag mRNA and SIV protease mRNA at different ratios (5:1, 10:1, 20:1, 40:1 and 80:1). The cells were harvested 48 hours later, lysed and analyzed by WB. Increasing levels of fully processed Gag p27 were detected using serial dilutions of protease with optimal balance between yield, processing and cell viability between 40:1 and 80:1 ratios. The presence of bands corresponding to incompletely processed or unprocessed Gag is expected in whole cell lysate because only a fraction of Gag is fully processed at any given time even in the presence of excess protease. The yield of Gag expression is lower at high protease concentrations due to poor cell viability. The WB was revealed using Rabbit anti-SIVp27 polyclonal antiserum at 1:2000 (SP1432-B) as a primary antibody and Donkey-anti-Rabbit antibodies, HRP-conjugated, at 1:5000 (R&D Systems) as a secondary antibody.

The data in this set of experiments shows that fully processed SIV core protein p27 is efficiently and selectively incorporated into virus-like particles (VLPs) in the presence of SIV protease (FIG. 7). The image in FIG. 7 shows a Western blot analysis of concentrated SHIV VLPs produced by 293-T human embryonic kidney cells stably transduced with HIV-1 WITO.153E Env and transfected with SIV Gag mRNA and SIV protease mRNA at different ratios (30:1, 40:1, 50:1). The culture supernatants were harvested 48 hours later, ultracentrifuged on a sucrose cushion to concentrate VLPs, and the pellets were analyzed by WB. Purified VLPs contain almost exclusively fully processed Gag. The WB was revealed using Rabbit anti-SIVp27 polyclonal antiserum at 1:2000 (SP1432-B) as a primary antibody and Donkey-anti-Rabbit antibodies, HRP-conjugated, at 1:5000 (R&D Systems) as a secondary antibody.

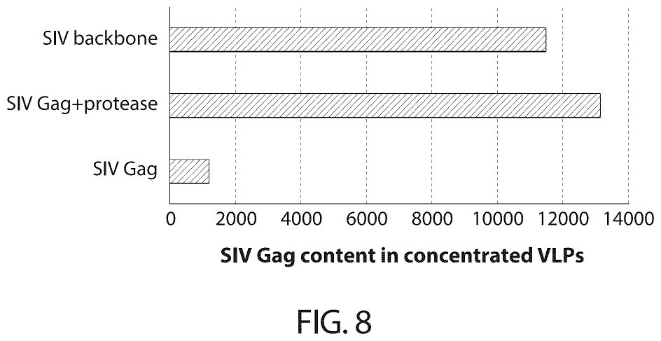

The data in this set of experiments shows that the production of extracellular SHIV VLPs is markedly increased in the presence of SIV protease (FIG. 8). The chart in FIG. 8 shows the results of quantitative ELISA measurements in concentrated VLPs obtained from 293-T cells stably expressing HIV-1 WITO.153E Env transfected with DNA plasmids expressing either SIV Gag alone, SIV-Gag+SIV protease or SIV full-length backbone (Env-deleted but containing protease). Culture supernatants were harvested 48 hours later, ultracentrifuged on a sucrose cushion to concentrate VLPs, and the pellets were analyzed by ELISA for SIV Gag p27 content.

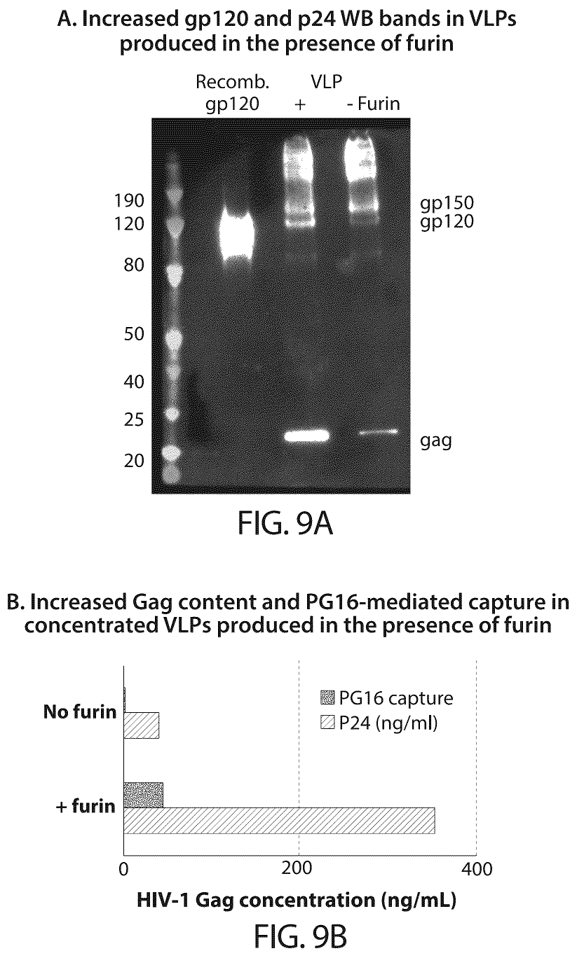

The data in this set of experiments shows that the efficiency of Env processing to gp120 (FIG. 9A) and the production of extracellular VLPs (FIG. 9B) are markedly increased in the presence of furin. FIG. 9A includes a Western blot showing the increased processing of the Env precursor gp150 to gp120 in the presence of furin. 293T cells were transfected with HIV-1 Env DNA and HIV Gag-Pol DNA with or without furin DNA. Culture supernatants were harvested 48 hours later, ultracentrifuged to concentrate VLPs, and pellets were lysed and analyzed by WB. The WB was revealed using Rabbit anti-HIV-1 Env polyclonal antibody at 1:200 (home made) as a primary antibody and Donkey-anti-Rabbit antibodies, HRP-conjugated, at 1:5000 (R&D Systems) as a secondary antibody. FIG. 9B includes a chart showing the results of quantitative ELISA measurements in concentrated VLPs obtained from 293T cells transfected with HIV-1 Env DNA and HIV Gag-Pol DNA with or without furin DNA. Culture supernatants were harvested 48 hours later and tested for total p24 content (green bars) or used for virion capture using mAb PG16 immobilized on magnetic beads (red bars). Captured virions were lysed and analyzed by ELISA for p24 content. The efficiency of PG16-mediated capture is typically low (between 10% and 20% of the total p24 content) even on infectious viral stocks, presumably due to the low density of Env on the virion surface and/or epitope masking by serum proteins.

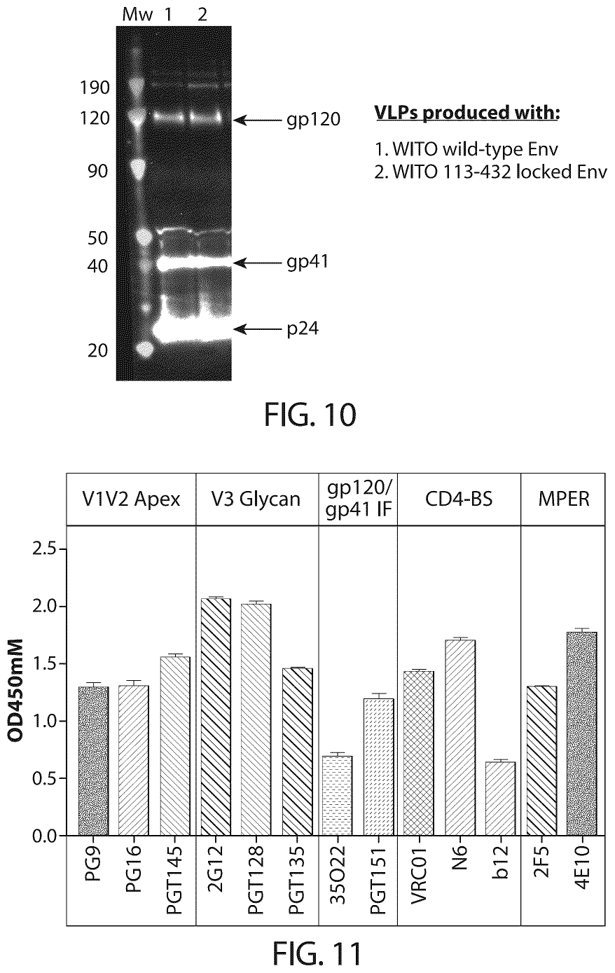

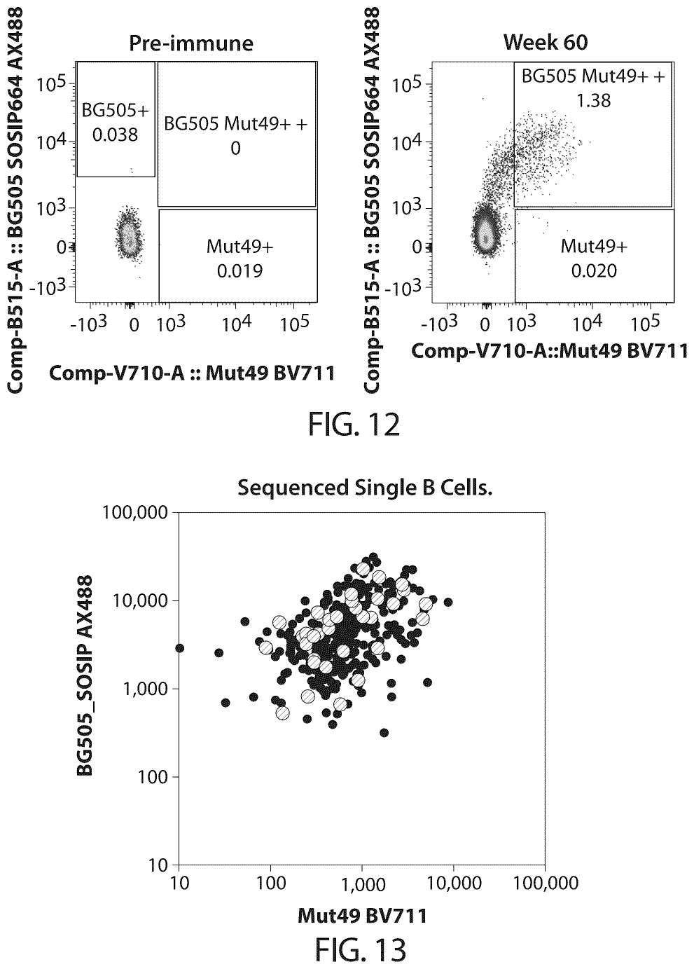

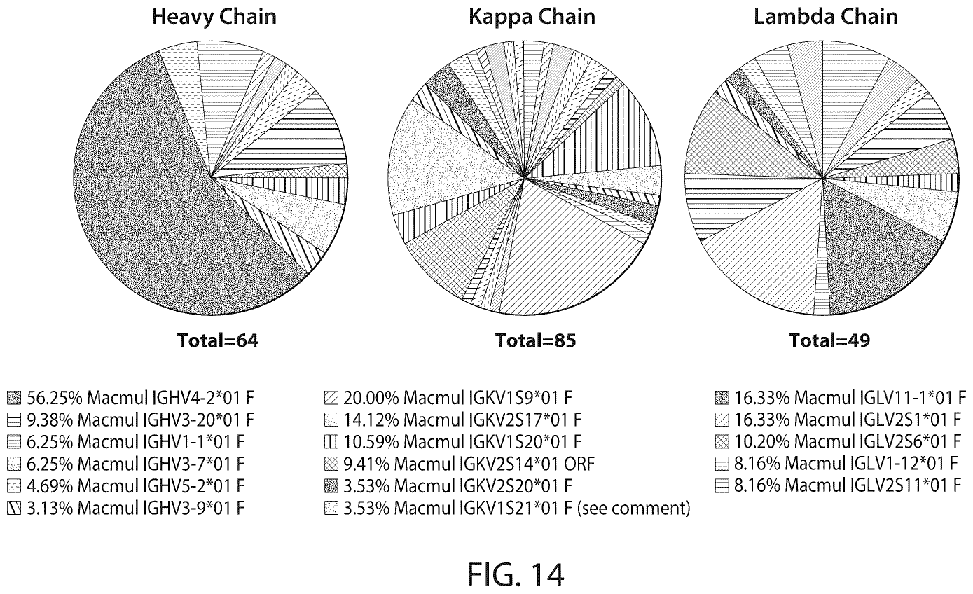

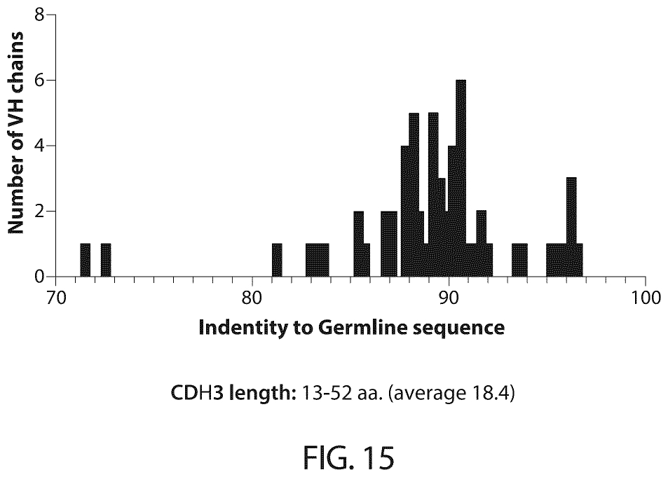

The data in this set of experiments shows that both HIV-1 Env and Gag are fully processed in virus-like particles (VLPs) produced using Gag-Pol (which expresses also the viral protease) (FIG. 10). The image in FIG. 10 shows a Western blot analysis of concentrated HIV-1 VLPs produced by 293-T human embryonic kidney cells stably transduced with HIV-1 WITO.153E wild-type or interdomain-locked (113-432) Env and transfected with HIV-1 Gag-Pol plasmid. The culture supernatants were harvested 48 hours later and VLPs were purified using magnetic beads coated with anti-Env mAb PG16. This selection enriches for VLPs with high Env content. Antibody-captured VLPs were then lysed and analyzed by WB using a polyclonal human HIV-positive serum as a primary antibody and human IgG antibodies, HRP-conjugated, as a secondary antibody.

The data in this set of experiments shows that VLPs produced using Gag-Pol display a desired vaccine-relevant antigenic profile, with high expression of epitopes recognized by broadly neutralizing antibodies (bNAbs) (FIG. 11). 293T cells stably expressing HIV-1 WITO.153E Env were transfected with HIV-1 Gag-Pol, which contains the viral protease gene. Culture supernatants were collected at 48 hrs., ultracentrifuged on a sucrose cushion to concentrate VLPs, and washed once with phosphate buffer saline. The expression of epitopes recognized by bNAbs was tested by VLP ELISA: briefly, the pelleted material was captured on ELISA plates using Galanthus nivalis lectin; after washing and blocking with 0.05% casein, different human bNAbs were added for 45 min at room temperature, washed and revealed using a goat-anti-human HRP-conjugated secondary antibody. The antigenic supersites recognized by the different groups of bNAbs are indicated at the top. IF=gp120/gp4l interface−CD4-BS=CD4 binding site−MPER=Membrane-proximal extracellular region of gp41.

### Example 3

In this example, we cloned a significant number of diverse antibodies specific for the CD4-binding site. A majority of them use VH4, which is the heavy chain used by all but 2 of the anti-CD4-binding site neutralizing antibodies cloned from macaques in another study (Mason et al. PLOS Pathogen, 2016, DOI:10.1371/journal.ppat.1005537).

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

1-22. (canceled)

23. A method of inducing in a subject an immune response to human immunodeficiency virus (HIV), the method comprising:
administering to the subject a first lipid nanoparticle comprising a messenger RNA (mRNA) encoding an HIV envelope (Env) protein from a first Clade and an mRNA encoding a lentivirus group-specific antigen (Gag) protein; and
administering to the subject a second lipid nanoparticle comprising a mRNA encoding an HIV Env protein from a second Clade and a mRNA encoding a lentivirus Gag protein,
wherein the first lipid nanoparticle and the second lipid nanoparticle are administered more than once and in an amount effective at inducing in the subject a population of neutralizing antibodies that bind to shared epitopes on proteins from the first Clade and neutralizing antibodies that bind to shared epitopes on proteins from the second Clade.

24-25. (canceled)

26. The method of claim 23 further comprising administering to the subject at least one additional lipid nanoparticle comprising an mRNA encoding an HIV Env protein from at least one additional Clade and a mRNA encoding an HIV Gag protein.

27. (canceled)

28. The method of claim 23, wherein the first nanoparticle comprises a ratio of the mRNA encoding an HIV Env protein to the mRNA encoding an HIV Gag protein of at least 1:1, and/or wherein the second nanoparticle comprises a ratio the mRNA encoding an HIV Env protein to the mRNA encoding an HIV Gag protein of at least 1:1.

29. The method of claim 28, wherein the first nanoparticle comprises a ratio of the mRNA encoding an HIV Env protein to the mRNA encoding an HIV Gag protein of at least 3:2, and/or wherein the second nanoparticle comprises a ratio the mRNA encoding an HIV Env protein to the mRNA encoding an HIV Gag protein of at least 3:2.

30. The method of claim 23, wherein the HIV Env protein comprises mutations, relative to wild-type HIV Env protein, that favor a closed conformation.

31. The method of claim 23, wherein the HIV Env protein comprises glycan knock-in or knock-out modifications.

32. The method of claim 23, wherein the HIV Env protein is a stabilized soluble Env protein.

33. The method of claim 32, wherein the HIV Env protein is an HIV Env SOSIP.664 protein.

34. The method of claim 23, wherein the HIV Env protein is a membrane-bound HIV Env protein.

35. The method of claim 34, wherein the membrane-bound HIV Env protein comprises a cytosolic portion, wherein the cytosolic portion of the protein is truncated.

36. The method of claim 34, wherein the membrane-bound HIV Env protein is gp150 or gp160.

37. The method of claim 23, wherein the lentivirus is selected from human immunodeficiency virus (HIV), simian immunodeficiency virus (SIV), and murine leukemia virus (muLV).

38. The method of claim 26, wherein the first Clade, the second Clade, and the at least one additional Clade are selected from the group consisting of HIV Group M Clades A-K and related circulating recombinant forms (CRFs).

39. The method of claim 23, wherein the first lipid nanoparticle and the second nanoparticle are administered sequentially.

40. The method of claim 39, wherein the first lipid nanoparticle is administered as multiple doses separated by at least 1 week per administration, prior to administration of the second lipid nanoparticle.

41.-66. (canceled)

67. The method of claim 23, wherein the HIV Env protein comprises a sequence of an Env protein of an HIV strain obtained from an infected subject who has broadly neutralizing antibodies to HIV Env protein.

68. The method of claim 23, wherein the HIV Env protein comprises a consensus sequence of variants an Env protein of an HIV strain obtained from an infected subject who has broadly neutralizing antibodies to HIV Env protein.

69. The method of claim 23, wherein the method comprises
administering to the subject a first lipid nanoparticle comprising a mRNA encoding a first HIV Env protein and a mRNA encoding an HIV Gag polyprotein;
administering to the subject a second lipid nanoparticle comprising a mRNA encoding a second HIV Env protein and a mRNA encoding an HIV Gag polyprotein; and
administering to the subject a third lipid nanoparticle comprising a mRNA encoding a third HIV Env protein and a mRNA encoding an HIV Gag polyprotein;
wherein the population of neutralizing antibodies comprises neutralizing antibodies that bind to shared epitopes on proteins from multiple different HIV strains.

70. The method of claim 69, wherein at least one of the first, second, and third HIV Env proteins comprises a sequence of an Env protein of an HIV strain obtained from an infected subject who has broadly neutralizing antibodies to HIV Env protein.

71. The method of claim 69, wherein at least one of the first, second, and third HIV Env proteins comprises a consensus sequence of variants an Env protein of an HIV strain obtained from an infected subject who has broadly neutralizing antibodies to HIV Env protein.

72. The method of claim 23, wherein the method comprises
administering to the subject a first lipid nanoparticle comprising a mRNA encoding an HIV Clade B Env protein and a mRNA encoding an HIV Gag polyprotein;
administering to the subject a second lipid nanoparticle comprising a mRNA encoding an HIV Clade A Env protein and a mRNA encoding an HIV Gag polyprotein; and
administering to the subject a third lipid nanoparticle comprising a mRNA encoding an HIV Clade C Env protein and a mRNA encoding an HIV Gag polyprotein;
wherein the population of neutralizing antibodies comprises neutralizing antibodies that bind to shared epitopes on HIV Clade B proteins, HIV Clade A proteins, and HIV Clade C proteins.

73-76. (canceled)

77. The method of claim 26, wherein none of the first, second, or at least one additional lipid nanoparticles comprise mRNA encoding a soluble HIV Env protein.

78-80. (canceled)
